SEQUENCE LISTING (96 sequences)

SEQ ID NO: 1 - protein, 7 aa, Homo sapiens
GITVSSN

SEQ ID NO: 2 - protein, 10 aa, Homo sapiens
GITVSSNYMT

SEQ ID NO: 3 - protein, 5 aa, Homo sapiens
SNYMT

SEQ ID NO: 4 - protein, 6 aa, Homo sapiens
SSNYMT

SEQ ID NO: 5 - protein, 8 aa, Homo sapiens
GITVSSNY

SEQ ID NO: 6 - protein, 5 aa, Homo sapiens
YSGGS

SEQ ID NO: 7 - protein, 9 aa, Homo sapiens
VIYSGGSTF

SEQ ID NO: 8 - protein, 16 aa, Homo sapiens
VIYSGGSTFYADSVRG

SEQ ID NO: 9 - protein, 12 aa, Homo sapiens
WVSVIYSGGSTF

SEQ ID NO: 10 - protein, 7 aa, Homo sapiens
IYSGGST

SEQ ID NO: 11 - protein, 10 aa, Homo sapiens
DLEMAGAFDI

SEQ ID NO: 12 - protein, 11 aa, Homo sapiens
ARDLEMAGAFD

SEQ ID NO: 13 - protein, 12 aa, Homo sapiens
ARDLEMAGAFDI

SEQ ID NO: 14 - protein, 12 aa, Homo sapiens
RASQSVSSSYLA

SEQ ID NO: 15 - protein, 8 aa, Homo sapiens
SSSYLAWY

SEQ ID NO: 16 - protein, 7 aa, Homo sapiens
QSVSSSY

SEQ ID NO: 17 - protein, 7 aa, Homo sapiens
GASSRAT

SEQ ID NO: 18 - protein, 10 aa, Homo sapiens
LLIYGASSRA

SEQ ID NO: 19 - protein, 8 aa, Homo sapiens
QQYGSSYT

SEQ ID NO: 20 - protein, 7 aa, Homo sapiens
QQYGSSY

SEQ ID NO: 21 - protein, 118 aa, Homo sapiens
EVQLVESGGGLVQPGGSLRLSCAASGITVSSNYMTWVRQAPGKGLEWVSVIYSGGSTFYADSVRGRFTISRDNSKNTLYLQMNSLRAEDTAVYYCARDLEMAGAFDIWGQGTMVTVSS

SEQ ID NO: 22 - protein, 107 aa, Homo sapiens
EIVLTQSPGTLSLSPGERATLSCRASQSVSSSYLAWYQQKPGQAPRLLIYGASSRATGIPDRFSGSGSGTDFTLTISRLEPEDSAVYYCQQYGSSYTFGQQTKLEIK

SEQ ID NO: 23 - protein, 7 aa, Homo sapiens
GYTFSSY

SEQ ID NO: 24 - protein, 10 aa, Homo sapiens
GYTFSSYGIS

SEQ ID NO: 25 - protein, 5 aa, Homo sapiens
SYGIS

SEQ ID NO: 26 - protein, 6 aa, Homo sapiens
SSYGIS

SEQ ID NO: 27 - protein, 8 aa, Homo sapiens
GYTFSSYG

SEQ ID NO: 28 - protein, 6 aa, Homo sapiens
SPYNGN

SEQ ID NO: 29 - protein, 10 aa, Homo sapiens
WISPYNGNTK

SEQ ID NO: 30 - protein, 17 aa, Homo sapiens
WISPYNGNTKYPQKFQG

SEQ ID NO: 31 - protein, 13 aa, Homo sapiens
WMGWISPYNGNTK

SEQ ID NO: 32 - protein, 8 aa, Homo sapiens
ISPYNGNT

SEQ ID NO: 33 - protein, 10 aa, Homo sapiens
DLELGGGFDY

SEQ ID NO: 34 - protein, 11 aa, Homo sapiens
ARDLELGGGFD

SEQ ID NO: 35 - protein, 12 aa, Homo sapiens
ARDLELGGGFDY

SEQ ID NO: 36 - protein, 14 aa, Homo sapiens
TGTSSDVGSYNLVS

SEQ ID NO: 37 - protein, 10 aa, Homo sapiens
VGSYNLVSWY

SEQ ID NO: 38 - protein, 9 aa, Homo sapiens
SSDVGSYNL

SEQ ID NO: 39 - protein, 7 aa, Homo sapiens
EGTKRPS

SEQ ID NO: 40 - protein, 10 aa, Homo sapiens
FMIYEGTKRP

SEQ ID NO: 41 - protein, 10 aa, Homo sapiens
CSYAGNSTWV

SEQ ID NO: 42 - protein, 9 aa, Homo sapiens
CSYAGNSTW

SEQ ID NO: 43 - protein, 119 aa, Homo sapiens
QVQLVQSGAEVKKPGASVKVSCKASGYTFSSYGISWVRQAPGQGLEWMGWISPYNGNTKYPQKFQGRVTMTTDTSTNTAYMELRSLRSDDTAVYYCARDLELGGGFDYWGQGTLVTVSS

SEQ ID NO: 44 - protein, 110 aa, Homo sapiens
QSALTQPASVSGSPGQSITISCTGTSSDVGSYNLVSWYQQHPDKAPKFMIYEGTKRPSGVSNRFSGSKSGNTASLTISGLQAEDEADYYCCSYAGNSTWVFGGGTKLTVL

SEQ ID NO: 45 - protein, 23 aa, Homo sapiens
EIVLTQSPGTLSLSPGERATLSC

SEQ ID NO: 46 - protein, 15 aa, Homo sapiens
WYQQKPGQAPRLLIY

SEQ ID NO: 47 - protein, 32 aa, Homo sapiens
GIPDRFSGSGSGTDFTLTISRLEPEDSAVYYC

SEQ ID NO: 48 - protein, 10 aa, Homo sapiens
FGQGTKLEIK

SEQ ID NO: 49 - protein, 25 aa, Homo sapiens [sequence omitted]

SEQ ID NO: 50 - protein, 19 aa, Homo sapiens
YMTWVRQAPGKGLEWVSVI

SEQ ID NO: 51 - protein, 41 aa, Homo sapiens
TFYADSVRGRFTISRDNSKNTLYLQMNSLRAEDTAVYYCAR

SEQ ID NO: 52 - protein, 11 aa, Homo sapiens
WGQGTMVTVSS

SEQ ID NO: 53 - protein, 22 aa, Homo sapiens
QSALTQPASVSGSPGQSITISC

SEQ ID NO: 54 - protein, 15 aa, Homo sapiens
WYQQHPDKAPKFMIY

SEQ ID NO: 55 - protein, 32 aa, Homo sapiens
GVSNRFSGSKSGNTASLTISGLQAEDEADYYC

SEQ ID NO: 56 - protein, 10 aa, Homo sapiens
FGGGTKLTVL

SEQ ID NO: 57 - protein, 25 aa, Homo sapiens [sequence omitted]

SEQ ID NO: 58 - protein, 19 aa, Homo sapiens
GISWVRQAPGQGLEWMGWI

SEQ ID NO: 59 - protein, 41 aa, Homo sapiens
TKYPQKFQGRVTMTTDTSTNTAYMELRSLRSDDTAVYYCAR

SEQ ID NO: 60 - protein, 11 aa, Homo sapiens
WGQGTLVTVSS

SEQ ID NO: 61 - protein, 29 aa, Homo sapiens
EIVLTQSPGTLSLSPGERATLSCRASQSV

SEQ ID NO: 62 - protein, 26 aa, Homo sapiens [sequence omitted]

SEQ ID NO: 63 - protein, 9 aa, Homo sapiens
QQKPGQAPR

SEQ ID NO: 64 - protein, 17 aa, Homo sapiens
LAWYQQKPGQAPRLLIY

SEQ ID NO: 65 - protein, 33 aa, Homo sapiens
TGIPDRFSGSGSGTDFTLTISRLEPEDSAVYYC

SEQ ID NO: 66 - protein, 37 aa, Homo sapiens
SSRATGIPDRFSGSGSGTDFTLTISRLEPEDSAVYYC

SEQ ID NO: 67 - protein, 11 aa, Homo sapiens
TFGQGTKLEIK

SEQ ID NO: 68 - protein, 30 aa, Homo sapiens
EVQLVESGGGLVQPGGSLRLSCAASGITVS

SEQ ID NO: 69 - protein, 29 aa, Homo sapiens
EVQLVESGGGLVQPGGSLRLSCAASGITV

SEQ ID NO: 70 - protein, 14 aa, Homo sapiens
WVRQAPGKGLEWVS

SEQ ID NO: 71 - protein, 11 aa, Homo sapiens
WVRQAPGKGLE

SEQ ID NO: 72 - protein, 17 aa, Homo sapiens
MTWVRQAPGKGLEWVSV

SEQ ID NO: 73 - protein, 39 aa, Homo sapiens
YADSVRGRFTISRDNSKNTLYLQMNSLRAEDTAVYYCAR

SEQ ID NO: 74 - protein, 32 aa, Homo sapiens
RFTISRDNSKNTLYLQMNSLRAEDTAVYYCAR

SEQ ID NO: 75 - protein, 37 aa, Homo sapiens
YADSVRGRFTISRDNSKNTLYLQMNSLRAEDTAVYYC

SEQ ID NO: 76 - protein, 38 aa, Homo sapiens
FYADSVRGRFTISRDNSKNTLYLQMNSLRAEDTAVYYC

SEQ ID NO: 77 - protein, 12 aa, Homo sapiens
IWGQGTMVTVSS

SEQ ID NO: 78 - protein, 28 aa, Homo sapiens
QSALTQPASVSGSPGQSITISCTGTSSD

SEQ ID NO: 79 - protein, 25 aa, Homo sapiens
QSALTQPASVSGSPGQSITISCTGT

SEQ ID NO: 80 - protein, 9 aa, Homo sapiens
QQHPDKAPK

SEQ ID NO: 81 - protein, 17 aa, Homo sapiens
VSWYQQHPDKAPKFMIY

SEQ ID NO: 82 - protein, 33 aa, Homo sapiens
SGVSNRFSGSKSGNTASLTISGLQAEDEADYYC

SEQ ID NO: 83 - protein, 37 aa, Homo sapiens
TKRPSGVSNRFSGSKSGNTASLTISGLQAEDEADYYC

SEQ ID NO: 84 - protein, 11 aa, Homo sapiens
VFGGGTKLTVL

SEQ ID NO: 85 - protein, 30 aa, Homo sapiens
QVQLVQSGAEVKKPGASVKVSCKASGYTFS

SEQ ID NO: 86 - protein, 29 aa, Homo sapiens
QVQLVQSGAEVKKPGASVKVSCKASGYTF

SEQ ID NO: 87 - protein, 14 aa, Homo sapiens
WVRQAPGQGLEWMG

SEQ ID NO: 88 - protein, 11 aa, Homo sapiens
WVRQAPGQGLE

SEQ ID NO: 89 - protein, 17 aa, Homo sapiens
ISWVRQAPGQGLEWMGW

SEQ ID NO: 90 - protein, 39 aa, Homo sapiens
YPQKFQGRVTMTTDTSTNTAYMELRSLRSDDTAVYYCAR

SEQ ID NO: 91 - protein, 32 aa, Homo sapiens
RVTMTTDTSTNTAYMELRSLRSDDTAVYYCAR

SEQ ID NO: 92 - protein, 37 aa, Homo sapiens
YPQKFQGRVTMTTDTSTNTAYMELRSLRSDDTAVYYC

SEQ ID NO: 93 - protein, 38 aa, Homo sapiens
KYPQKFQGRVTMTTDTSTNTAYMELRSLRSDDTAVYYC

SEQ ID NO: 94 - protein, 12 aa, Homo sapiens
YWGQGTLVTVSS

SEQ ID NO: 95 - DNA, 40 bp, synthetic construct
  source 1..40: mol_type = other DNA
ctacacgacgctcttccgatctagcagtggtatcaacgca

SEQ ID NO: 96 - DNA, 25 bp, synthetic construct
  source 1..25: mol_type = other DNA
ctacacgacgctcttccgatctagc

(19) United States
(12) Patent Application Publication
(10) Pub. No.: US 2025/0319125 A1
(43) Pub. Date: Oct. 16, 2025

(54) NEUTRALIZING ANTIBODIES AGAINST SARS-COV-2 AND USES THEREOF

(71) Applicants: VAL-CHUM, LIMITED PARTNERSHIP, Québec (CA); VALORISATION HSJ, LIMITED PARTNERSHIP, Québec (CA)

(72) Inventors: Andrés FINZI, Saint-Bruno-de-Montarville (CA); Elie HADDAD, Hampstead (CA); Martin SMITH, Montréal (CA); Vincent-Philippe LAVALLÉE, Montréal (CA); Véronique LISI, Verdun (CA); Jérémie PRÉVOST, Mirabel (CA)

(21) Appl. No.: 18/576,567

(22) PCT Filed: Jul. 9, 2022
(86) PCT No.: PCT/CA2022/051074

§ 371 Date: Jan. 4, 2024

Related U.S. Application Data
(60) Provisional application No. 63/203,126, filed on Jul. 9, 2021.

(51) Int. Cl. A61K 35/17; A61K 40/15; A61K 40/31; A61K 40/46; A61P 31/14; C07K 16/10

(52) U.S. Cl. CPC A61K 35/17; A61K 40/15; A61K 40/31; A61K 40/46; A61P 31/14; C07K 16/1003; A61K 2239/13; C07K 2317/565; C07K 2317/567; C07K 2317/76

(57) ABSTRACT

The present application discloses neutralizing anti-SARS-COV-2 antibodies or antigen-binding fragments thereof that are effective against several SARS-COV-2 variants. Pharmaceutical compositions comprising the antibodies or antigen-binding fragments thereof are also disclosed. The antibodies, antigen-binding fragments thereof, or pharmaceutical compositions described herein may be used for the prevention and/or treatment of coronavirus infection and/or associated diseases and symptoms, such as SARS-COV-2 infection and/or COVID-19.

### CROSS REFERENCE TO RELATED APPLICATIONS

The present application is a National Entry Application of PCT application No. PCT/CA2022/051074 filed on Jul. 9, 2022, which itself claims benefit of U.S. provisional patent application No. 63/203,126 filed on Jul. 9, 2021. All documents above are incorporated herein in their entirety by reference.

### TECHNICAL FIELD

The present disclosure generally relates to viral infections, and more particularly to the prevention and/or treatment of coronavirus infection and related diseases, such as severe acute respiratory syndrome coronavirus 2 (SARS-COV-2) infection and COVID-19.

### BACKGROUND ART

Coronaviruses are large, roughly spherical, RNA viruses with bulbous surface projections that cause diseases in mammals and birds. In humans, these viruses cause respiratory tract infections that can range from mild to lethal. Mild illnesses include some cases of the common cold (which is also caused by other viruses, predominantly rhinoviruses), while more lethal varieties can cause severe acute respiratory syndrome (SARS), Middle East respiratory syndrome (MERS), and Coronavirus disease 2019 (COVID-19). Coronaviruses have four structural proteins, namely the Spike(S), Envelope (E), and Membrane (M) proteins, forming the viral envelope, as well as the Nucleocapsid (N) protein, holding the viral RNA genome

Severe acute respiratory syndrome coronavirus 2 (SARS-COV-2) is the strain of coronavirus that causes COVID-19, the respiratory illness responsible for the COVID-19 pandemic. The spike protein SARS-COV-2 is the glycoprotein responsible for allowing the virus to attach to and fuse with the membrane of a host cell; specifically, its S1 subunit catalyzes attachment, the S2 subunit fusion. The main receptor involved in SARS-COV-2 entry into human cells is the angiotensin converting enzyme 2 (ACE2). After attachment of a SARS-COV-2 virion to a target cell, the cell's protease transmembrane protease, serine 2 (TMPRSS2) cuts open the spike protein of the virus, exposing a fusion peptide in the S2 subunit, and the host receptor ACE2.

Multiple variants of SARS-COV-2 are circulating globally and within the United States. Four new variants that have rapidly become dominant within their countries have aroused concerns: B.1.1.7 (also known as VOC-202012/01), 501Y.V2 (B.1.351), P.1 (B.1.1.28.1) and delta (B.1.617.2).

The B.1.1.7 variant (23 mutations with 17 amino acid changes) was first described in the United Kingdom in December 2020; the 501Y.V2 variant (23 mutations with 17 amino acid changes) was initially reported in South Africa in December 2020; and the P.1 variant (approximately 35 mutations with 17 amino acid changes) was reported in Brazil in January 2021. By February 2021, the B.1.1.7 variant had been reported in 93 countries, the 501Y.V2 variant in 45, and the P.1 variant in 21. All three variants have the N501Y mutation, which changes the amino acid asparagine (N) to tyrosine (Y) at position 501 in the receptor-binding domain of the spike protein. The 501Y.V2 and P.1 variants both have two additional receptor-binding-domain mutations, K417N/T and E484K. These mutations increase the binding affinity of the receptor-binding domain to the angiotensin-converting enzyme 2 (ACE2) receptor. Four key concerns stemming from the emergence of the new variants are their effects on viral transmissibility, disease severity, reinfection rates (i.e., escape from natural immunity), and vaccine effectiveness (i.e., escape from vaccine-induced immunity). Recently, two more SARS-COV-2 variants, B.1.427 and B.1.429, which were first detected in California, have been shown to be approximately 20% more transmissible than pre-existing variants and have been classified by the CDC as variants of concern. The B.1.617.2 delta variant comprises the following substitutions in the Spike protein that are known to affect transmissibility of the virus: D614G, T478K, P681R and L452R. Studies on these variants have provided compelling evidence that they have the potential to escape naturally-induced immunity as well as the immunity induced by currently approved vaccines.

Current evidence indicates that SARS-COV-2, the etiologic agent of COVID-19, will become endemic in the population. The current pandemic is aggravated by the apparition of variants of concern that are feared to result in an antigenic drift that could evade vaccine-elicited immune responses.

Thus, there is a need for the development of therapies that elicit neutralizing activity against SARS-COV-2, including SARS-COV-2 variants.

The present description refers to a number of documents, the content of which is herein incorporated by reference in their entirety.

### SUMMARY

The present disclosure provides the following items 1 to 61:
1. An antibody or an antigen binding fragment thereof comprises one of the following combinations of complementarity determining regions (CDRs):
(a) a light chain CDR1 (CDR-L1) comprising an amino acid sequence having at least 70% identity with the sequence RASQSVSSSYLA (SEQ ID NO:14); a CDR-L2 comprising an amino acid sequence having at least 70% identity with the sequence GASSRAT (SEQ ID NO:17); a CDR-L3 comprising an amino acid sequence having at least 70% identity with the sequence QQYGSSYT (SEQ ID NO:19); a heavy chain CDR1 (CDR-H1) comprising an amino acid sequence having at least 70% identity with the sequence GITVSSN (SEQ ID NO:1); a CDR-H2 comprising an amino acid sequence having at least 70% identity with the sequence YSGGS (SEQ ID NO:6); and a CDR-H3 comprising an amino acid sequence having at least 70% identity with the sequence DLEMAGAFDI (SEQ ID NO:11); or
(b) a CDR-L1 comprising an amino acid sequence having at least 70% identity with the sequence TGTSSDVGSYNLVS (SEQ ID NO:36); a CDR-L2 comprising an amino acid sequence having at least 70% identity with the sequence EGTKRPS (SEQ ID NO:39); a CDR-L3 comprising an amino acid sequence having at least 70% identity with the sequence CSYAGNSTWV (SEQ ID NO:41); a CDR-H1 comprising an amino acid sequence having at least 70% identity with the sequence GYTFSSY (SEQ ID NO:23); a CDR-H2 comprising an amino acid sequence having at least 70% identity with the sequence SPYNGN (SEQ ID NO:28); and a CDR-H3 comprising an amino acid sequence having at least 70% identity with the sequence DLELGGGFDY (SEQ ID NO:33).
2. The antibody or antigen binding fragment thereof of item 1, which comprises the following combinations of complementarity determining regions (CDRs):
   a CDR-L1 comprising the sequence RASQSVSSSYLA (SEQ ID NO:14); a CDR-L2 comprising the sequence GASSRAT (SEQ ID NO:17); a CDR-L3 comprising the sequence QQYGSSYT (SEQ ID NO:19); a CDR-H1 comprising the sequence GITVSSN (SEQ ID NO:1); a CDR-H2 comprising the sequence YSGGS (SEQ ID NO:6); and a CDR-H3 comprising the sequence DLEMAGAFDI (SEQ ID NO:11).
3. The antibody or antigen binding fragment thereof of item 1, which comprises the following combinations of complementarity determining regions (CDRs):
   a CDR-L1 comprising the sequence TGTSSDVGSYNLVS (SEQ ID NO:36); a CDR-L2 comprising the sequence EGTKRPS (SEQ ID NO:39); a CDR-L3 comprising the sequence CSYAGNSTWV (SEQ ID NO:41); a CDR-H1 comprising the sequence GYTFSSY (SEQ ID NO: 23); a CDR-H2 comprising the sequence SPYNGN (SEQ ID NO:28); and a CDR-H3 comprising the sequence DLELGGGFDY (SEQ ID NO:33).
4 The antibody or antigen binding fragment thereof of any one of items 1 to 3, which further comprises the following light chain framework regions (FRs):
   (i) a light chain FR1 comprising an amino acid sequence having at least 50% identity with the sequence EIVLTQSPGTLSLSPGERATLSC (SEQ ID NO:45) or QSALTQPASVSGSPGQSITISC (SEQ ID NO:53); (ii) a light chain FR2 comprising an amino acid sequence having at least 50% identity with the sequence WYQQKPGQAPRLLIY (SEQ ID NO:46) or WYQQHPDKAPKFMIY (SEQ ID NO:54); (iii) a light chain FR3 comprising an amino acid sequence having at least 50% identity with the sequence GIPDRFSGSGSGTDFTLTISRLEPEDSAVYYC (SEQ ID NO:47) or GVSNRFSGSKSGNTASLTISGLQAEDEADYYC (SEQ ID NO:55); (iv) a light chain FR4 comprising an amino acid sequence having at least 50% identity with the sequence FGQGTKLEIK (SEQ ID NO:48) or FGGGTKLTVL (SEQ ID NO:56); or (v) any combination of (i) to (iv).
5. The antibody or antigen binding fragment thereof of item 4, which comprises the following FRs:
   (i) a light chain FR1 comprising the sequence EIVLTQSPGTLSLSPGERATLSC (SEQ ID NO: 45); (ii) a light chain FR2 comprising the sequence WYQQKPGQAPRLLIY (SEQ ID NO:46); (iii) a light chain FR3 comprising the sequence GIPDRFSGSGSGTDFTLTISRLEPEDSAVYYC (SEQ ID NO:47); and (iv) a light chain FR4 comprising the sequence FGQGTKLEIK (SEQ ID NO: 48).
6. The antibody or antigen binding fragment thereof of item 4, which comprises the following light chain FRs:
   (i) a light chain FR1 comprising the sequence QSALTQPASVSGSPGQSITISC (SEQ ID NO: 53); (ii) a light chain FR2 comprising the sequence WYQQHPDKAPKFMIY (SEQ ID NO:54); (iii) a light chain FR3 comprising the sequence GVSNRFSGSKSGNTASLTISGLQAEDEADYYC

(SEQ ID NO:55); and (iv) a light chain FR4 comprising the sequence FGGGTKLTVL (SEQ ID NO: 56).
7. The antibody or antigen binding fragment thereof of any one of items 1 to 6, which further comprises the following heavy chain FRs:
   (i) a heavy chain FR1 comprising an amino acid sequence having at least 50% identity EVOLVESGGGLVQPGGSLRLSCAAS with the sequence (SEQ ID NO:49) or QVQLVQSGAEVKKPGASVKVSCKAS (SEQ ID NO:57); (ii) a heavy chain FR2 comprising an amino acid sequence having at least 50% with identity the sequence YMTWVRQAPGKGLEWVSVI (SEQ ID NO:50) or GISWVRQAPGQGLEWMGWI (SEQ ID NO: 58); (iii) a heavy chain FR3 comprising an amino acid sequence having at least 50% identity with the sequence TFYADSVRGRFTISRDNSKNTLYLQMNSLRAEDTAVYYCAR (SEQ ID NO: 51) or TKYPQKFQGRVTMTTDTSTNTAYMELRSLRSDDTAVYYCAR (SEQ ID NO:59); (iv) a heavy chain FR4 comprising an amino acid sequence having at least 50%, 60%, 70%, 75%, 80%, 85%, 90% or 95% identity with the sequence WGQGTMVTVSS (SEQ ID NO:52) or WGQGTLVTVSS (SEQ ID NO:60); or (v) any combination of (i) to (iv).
8. The antibody or antigen binding fragment thereof of item 7, which further comprises the following heavy chain FRs:
   (i) a heavy chain FR1 comprising the sequence EVOLVESGGGLVQPGGSLRLSCAAS (SEQ ID NO:49); (ii) a heavy chain FR2 comprising the sequence GISWVRQAPGQGLEWMGWI (SEQ ID NO:50); (iii) a heavy chain FR3 comprising the sequence TKYPQKFQGRVTMTTDTSTNTAYMELRSLRSDDTAVYYCAR (SEQ ID NO:51); and (iv) a heavy chain FR4 comprising the sequence WGQGTMVTVSS (SEQ ID NO:52).
9. The antibody or antigen binding fragment thereof of item 7, which further comprises the following heavy chain FRs:
   (i) a heavy chain FR1 comprising the sequence QVQLVQSGAEVKKPGASVKVSCKAS (SEQ ID NO:57); (ii) a heavy chain FR2 comprising the sequence YMTWVRQAPGKGLEWVSVI (SEQ ID NO:58); (iii) a heavy chain FR3 comprising the sequence TFYADSVRGRFTISRDNSKNTLYLQMNSLRAEDTAVYYCAR (SEQ ID NO:59); and (iv) a heavy chain FR4 comprising the sequence WGQGTLVTVSS (SEQ ID NO:60).
10. The antibody or antigen binding fragment thereof of any one of items 1 to 9, which comprises a variable light chain comprising an amino acid sequence having at least 70% identity with the sequence EIVLTQSPGTLSLSPGERATLSCRASQSVSSSYLAWYQQKPGQAPRLLIYGASSRATGIPDRFS GSGSGTDFTLTISRLEPEDSAVYYCQQYGSSYTFGQQTKLEIK (SEQ ID NO:22); or QSALTQPASVSGSPGQSITISCTGTSSDVGSYNLVSWYQQHPDKAPKFMIYEGTKRPSGVSNR FSGSKSGNTASLTISGLQAEDEADYYCCSYAGNSTWVFGGGTKLTVL (SEQ ID NO:44).
11. The antibody or antigen binding fragment thereof of item 10, which comprises a variable light chain comprising the following sequence:

(SEQ ID NO: 22)
EIVLTQSPGTLSLSPGERATLSCRASQSVSSSYLAWYQQKPGQAPRLL
IYGASSRATGIPDRFSGSGSGTDFTLTISRLEPEDSAVYYCQQYGSSY
TFGQQTKLEIK.

12. The antibody or antigen binding fragment thereof of item 10, which comprises a variable 20 light chain comprising the following sequence:

(SEQ ID NO: 44)
QSALTQPASVSGSPGQSITISCTGTSSDVGSYNLVSWYQQHPDKAPKF
MIYEGTKRPSGVSNRFSGSKSGNTASLTISGLQAEDEADYYCCSYAGN
STWVFGGGTKLTVL.

13. The antibody or antigen binding fragment thereof of any one of items 1 to 12, which comprises a variable heavy chain comprising an amino acid sequence having at least 70% identity with the sequence:

(SEQ ID NO: 21)
EVQLVESGGGLVQPGGSLRLSCAASGITVSSNYMTWVRQAPGKGLEWV
SVIYSGGSTFYADSVRGRFTISRDNSKNTLYLQMNSLRAEDTAVYYCA
RDLEMAGAFDIWGQGTMVTVSS;
or
(SEQ ID NO: 43)
QVQLVQSGAEVKKPGASVKVSCKASGYTFSSYGISWVRQAPGQGLEWM
GWISPYNGNTKYPQKFQGRVTMTTDTSTNTAYMELRSLRSDDTAVYYC
ARDLELGGGFDYWGQGTLVTVSS.

14. The antibody or antigen binding fragment thereof of item 13, which comprises a variable heavy chain comprising the following sequence:

(SEQ ID NO: 21)
EVQLVESGGGLVQPGGSLRLSCAASGITVSSNYMTWVRQAPGKGLEWV
SVIYSGGSTFYADSVRGRFTISRDNSKNTLYLQMNSLRAEDTAVYYCA
RDLEMAGAFDIWGQGTMVTVSS.

15. The antibody or antigen binding fragment thereof of item 13, which comprises a variable heaw chain comprising the following sequence.

(SEQ ID NO: 43)
QVQLVQSGAEVKKPGASVKVSCKASGYTFSSYGISWVRQAPGQGLEWM
GWISPYNGNTKYPQKFQGRVTMTTDTSTNTAYMELRSLRSDDTAVYYC
ARDLELGGGFDYWGQGTLVTVSS.

16. A conjugate or a chimeric antigen receptor (CAR) comprising the antibody or antigen binding fragment thereof of any one of items 1 to 15.
17. A nucleic acid comprising a sequence encoding the light and/or heavy chain of the antibody or antigen binding fragment thereof of any one of items 1 to 15, or the CAR of item 16.
18. A host cell comprising the nucleic acid of item 17.
19. A pharmaceutical composition comprising the antibody or antigen binding fragment thereof of any one of items 1 to 15, the conjugate of item 16, or the cell of item 18, and a pharmaceutically acceptable excipient.
20. The pharmaceutical composition of item 19, wherein the pharmaceutical composition is in the form of an aerosol or an injectable solution.
21. A method for preventing or treating a SARS-COV-2 infection or a related disease (COVID-19) in a subject in need thereof, the method comprising administering to the subject an effective amount of the antibody or antigen-binding fragment thereof of any one of items 1 to 15, the conjugate of item 16, the cell of item 18, or the pharmaceutical composition of item 19 or 20.
22. A method for reducing the risk of developing Coronavirus disease 2019 (COVID-19) or the severity of COVID-19 in a subject infected by SARS-COV-2, the method comprising administering to the subject an effective amount of the antibody or antigen-binding fragment thereof of any one of items 1 to 15, or the pharmaceutical composition of item 19 or 20.
23. A method for blocking the entry of SARS-COV-2 in an ACE2-expressing cell, the method comprising contacting the cell and/or the virus with an effective amount of the antibody or antigen-binding fragment thereof of any one of items 1 to 15, the conjugate of item 16, the cell of item 18, or the pharmaceutical composition of item 19 or 20.
24. The method of any one of items 21 to 23, wherein the SARS-COV-2 is a variant of the Wuhan original SARS-COV-2 strain.
25. The method of any one of items 21 to 24, wherein the antibody, antigen-binding fragment thereof, or pharmaceutical composition is administered with at least one additional anti-SARS-CoV-2 antibody or antigen-binding fragment thereof.
26. Use of the antibody or antigen-binding fragment thereof of any one of items 1 to 15, the conjugate of item 16, the cell of item 18, or the pharmaceutical composition of item 19 or 20 for preventing or treating SARS-COV-2 infection or Coronavirus disease 2019 (COVID-19) in a subject.
27. Use of the antibody or antigen-binding fragment thereof of any one of items 1 to 15, the conjugate of item 16, the cell of item 18, or the pharmaceutical composition of item 19 or 20 for the manufacture of a medicament for preventing or treating SARS-COV-2 infection or Coronavirus disease 2019 (COVID-19) in a subject.
28. Use of the antibody or antigen-binding fragment thereof of any one of items 1 to 15, the conjugate of item 16, the cell of item 18, or the pharmaceutical composition of item 19 or 20 for reducing the risk of developing Coronavirus disease 2019 (COVID-19) or the severity of COVID-19 in a subject infected by SARS-COV-2.
29. Use of the antibody or antigen-binding fragment thereof of any one of items 1 to 15, the conjugate of item 16, the cell of item 18, or the pharmaceutical composition of item 19 or 20 for the manufacture of a medicament for reducing the risk of developing Coronavirus disease 2019(COVID-19) or the severity of COVID-19 in a subject infected by SARS-COV-2.
30. The use of any one of items 26 to 29, wherein the SARS-COV-2 is a variant of the Wuhan original SARS-COV-2 strain.
31. The use of any one of items 26 to 30, wherein the cell, antibody, antigen-binding fragment thereof, or pharmaceutical composition is for use with at least one additional anti-SARS-COV-2antibody or antigen-binding fragment thereof.
32. The antibody or antigen-binding fragment thereof of any one of items 1 to 15, the conjugate of item 16, the cell of item 18, or the pharmaceutical composition of item 19 or 20 for use in preventing or treating SARS-COV-2 infection or Coronavirus disease 2019 (COVID-19) in a subject.
33. The antibody or antigen-binding fragment thereof of any one of items 1 to 15, the conjugate of item 16, the cell of item 18, or the pharmaceutical composition of item 19 or 20 for use in reducing the risk of developing Coronavirus disease 2019 (COVID-19) or the severity of COVID-19 in a subject.
34. The antibody, antigen-binding fragment thereof, conjugate, cell or pharmaceutical composition for use according to item 32 or 33, wherein the SARS-COV-2 is a variant of the Wuhan original SARS-COV-2 strain.
35. The antibody, antigen-binding fragment thereof, conjugate, cell or pharmaceutical composition for use according to any one of items 32 to 34, wherein the antibody, antigen-binding fragment thereof, conjugate, cell or pharmaceutical composition is for use with at least one additional anti-SARS-COV-2 antibody or antigen-binding fragment thereof.
36. A recombinant antibody or antigen binding fragment thereof comprising:
(a) a heavy chain CDR1 comprising an amino acid sequence set forth in any one of SEQ ID NOs: 1 to 5;
(b) a heavy chain CDR2 comprising an amino acid sequence set forth in any one of SEQ ID NOs: 6 to 10;
(c) a heavy chain CDR3 comprising an amino acid sequence set forth in any one of SEQ ID NOs: 11 to 13;
(d) a light chain CDR1 comprising an amino acid sequence set forth in any one of SEQ ID NOs: 14 to 16;
(e) a light chain CDR2 comprising an amino acid sequence set forth in any one of SEQ ID NOs: 17, 18 and GA; and/or
(f) a light chain CDR3 comprising an amino acid sequence set forth in any one of SEQ ID NOs: 19 and 20.
37 The recombinant antibody or antigen binding fragment thereof of item 36, wherein the antibody is an IgG antibody.
38. The recombinant antibody or antigen binding fragment thereof of item 36, wherein the recombinant antibody or antigen binding fragment thereof is a Fab, F(ab)2, or a single chain variable fragment (scFv).
39. The recombinant antibody or antigen binding fragment thereof of item 36, wherein the recombinant antibody or antigen binding fragment thereof is chimeric or humanized.
40. The recombinant antibody of any one of items 36 to 39, wherein the antibody comprises an immunoglobulin heavy chain comprising an amino acid sequence at least about 90%, 95%, 97%, 98%, 99% or is identical to SEQ ID NO 21; and wherein the antibody comprises an immunoglobulin light chain comprising an amino acid sequence at least about 90%, 95%, 97%, 98%, 99% or is identical to SEQ ID NO 22.
41. A recombinant antibody or antigen binding fragment thereof comprising:
(a) a heavy chain CDR1 comprising an amino acid sequence set forth in any one of SEQ ID NOs: 23 to 27;
(b) a heavy chain CDR2 comprising an amino acid sequence set forth in any one of SEQ ID NOs: 28 to 32;
(c) a heavy chain CDR3 comprising an amino acid sequence set forth in any one of SEQ ID NOs: 33 to 35;
(d) a light chain CDR1 comprising an amino acid sequence set forth in any one of SEQ ID NOs: 36 to 38;
(e) a light chain CDR2 comprising an amino acid sequence set forth in any one of SEQ ID NOs: 39, 40 and EG; and/or
(f) a light chain CDR3 comprising an amino acid sequence set forth in any one of SEQ ID NOs: 41 and 42.
42. The recombinant antibody or antigen binding fragment thereof of item 41, wherein the antibody is an IgG antibody.
43. The recombinant antibody or antigen binding fragment thereof of item 41, wherein the recombinant antibody or antigen binding fragment thereof is a Fab, F (ab) 2, or a single chain variable fragment (scFv).
44. The recombinant antibody or antigen binding fragment thereof of item 41, wherein the recombinant antibody or antigen binding fragment thereof is chimeric or humanized.
45. The recombinant antibody of any one of items 41 to 44, wherein the antibody comprises an immunoglobulin heavy chain comprising an amino acid sequence at least about 90%, 95%, 97%, 98%, 99% or is identical to SEQ ID NO:43 and wherein the antibody comprises an immunoglobulin light chain comprising an amino acid sequence at least about 90%, 95%, 97%, 98%, 99% or is identical to SEQ ID NO:44.
46. The recombinant antibody or antigen binding fragment thereof of any one of items 36 to 45, wherein the antibody inhibits entry of SARS-COV-2 into a human cell.
47. The recombinant antibody or antigen binding fragment thereof of any one of items 36 to 46, wherein the antibody binds to the SARS-COV-2 RBD.
48. A nucleic acid encoding the recombinant antibody or antigen binding fragment thereof of any one of items 36 to 47.
49. A cell line comprising the nucleic acid of item 48.
50. The cell line of item 49, wherein the cell line is a Chinese Hamster Ovary (CHO) cell line.
51. A pharmaceutical composition comprising the recombinant antibody or antigen binding fragment thereof of any one of items 36 to 47 and a pharmaceutically acceptable excipient, carrier, or diluent.
52. The pharmaceutical composition of item 51, formulated for intravenous administration.
53. The pharmaceutical composition of item 51, formulated for administration by inhalation.
54. The pharmaceutical composition of item 51, formulated for administration by a nebulizer.
55. The recombinant antibody or antigen binding fragment thereof of any one of items 36 to 47 or the pharmaceutical composition of any one of items 51 to 54 for use in inhibiting entry of SARS-COV-2 into a human cell.
56. The recombinant antibody or antigen binding fragment thereof of any one of items 36 to 47 or the pharmaceutical composition of any one of items 51 to 54 for use in lessening the severity of a SARS-COV-2 infection or preventing severe SARS-COV-2 infection.
57. The recombinant antibody or antigen binding fragment thereof of any one of items 36 to 47 or the pharmaceutical composition of any one of items 51 to 54 for use in preventing or reducing the risk of death in an individual with acute respiratory distress caused by a SARS-COV-2 infection.
58. A method of lessening the severity of a SARS-COV-2 infection in an individual comprising administering to the individual a therapeutically effective amount of the recombinant antibody or antigen binding fragment thereof of any one of items 36 to 47 or the pharmaceutical composition of any one of items 51 to 54, thereby lessening the severity of a SARS-COV-2 infection.
59. A method of preventing severe SARS-COV-2 infection in an individual comprising administering to the individual a therapeutically effective amount of the recombinant antibody or antigen binding fragment thereof of any one of items 36 to 47 or the pharmaceutical composition of any one of items 51 to 54. thereby preventing severe SARS-COV-2 infection in an individual.
60. A method of preventing or reducing the risk of death in an individual with acute respiratory distress caused by a SARS-COV-2 infection comprising administering to the individual a therapeutically effective amount of the recombinant antibody or antigen binding fragment thereof of any one of items 36 to 47 or the pharmaceutical composition of any one of items 51 to 54, thereby preventing or reducing the risk of death in the individual with acute respiratory distress.
61. A method of inhibiting entry of SARS-COV-2 into a human cell in an individual comprising administering to the individual a therapeutically effective amount of the recombinant antibody or antigen binding fragment thereof of any one of items 36 to 47 or the pharmaceutical composition of any one of items 51 to 54, thereby inhibiting entry of the SARS-COV-2 into the human cell.

Other objects, advantages and features of the present invention will become more apparent upon reading of the following non-restrictive description of specific embodiments thereof, given by way of example only with reference to the accompanying drawings.

### BRIEF DESCRIPTION OF DRAWINGS

In the appended drawings:

FIGS. 1A-D show the isolation of RBD-specific mAbs from a pediatric patient. FIG. 1A: Indirect ELISA was performed using recombinant SARS-COV-2 RBD protein and incubation with COVID-19+ plasma samples from two adult donors (S002, S006 [CV3]) and one pediatric donor (Patient 12). Anti-RBD antibody binding was detected using horseradish peroxidase (HRP)-conjugated anti-human IgG. Relative light unit (RLU) values obtained with BSA (negative control) were subtracted and further normalized to the signal obtained with the anti-RBD CR3022 monoclonal antibody (mAb) present in each plate. Seropositivity threshold were calculated using ten pre-pandemic COVID-19-negative plasma samples. FIG. 1B: Pseudoviruses encoding the luciferase gene (Luc+) and bearing SARS-COV-2 full-length S (Wuhan-Hu-1 strain) were used to infect 293T-hACE2 cells in presence of increasing dilutions of indicated COVID-19+ plasma samples at 37° C. for 1 h prior infection of 293T-hACE2 cells. Fitted curves and half maximal inhibitory dilution (ID₅₀) values were determined using a normalized nonlinear regression. Error bars indicate means±SEM. FIG. 1C: Cryopreserved PBMCs obtained from Patient 12 were stained for the expression of cell-surface markers (CD3, CD14, CD19, IgD, IgG) and probed with fluorescently-labelled SARS-COV-2 RBD proteins. RBD-specific B cells (CD3−CD14−CD19+ IgD-IgG+RBD-AF488+RBD-AF647+) were individually sorted in a 96-well plate, followed by BCR sequencing. FIG. 1D: Pseudoviruses Luc+ bearing SARS-COV-2 full-length S (Wuhan-Hu-1 strain) were used to infect 293T-hACE2 cells in presence of increasing concentrations of indicated mAbs isolated from Patient 12 at 37° C. for 1 h prior infection of 293T-hACE2 cells. Fitted curves and half maximal inhibitory antibody concentration (IC₅₀) values were determined using a normalized nonlinear regression. Error bars indicate means±SEM .

FIGS. 2A and 2B show the amino acid sequences of the light and heavy chains of antibodies #3 and #8 described herein.

FIGS. 3A-G show the characterization of antibody #3 and antibody #8. FIGS. 3A-B: Cell-surface staining of 293T cells expressing full-length S from indicated variants using EH3 (FIG. 3A) and EH8 (FIG. 3B) monoclonal Abs (mAbs). The graphs show the median fluorescence intensities (MFI). Dashed lines indicate the reference value obtained with S D614G. Statistical significance was tested using mixed-effects ANOVA with a Dunnett post-test (****, p <0.0001). FIGS. 3C-D: Pseudoviruses encoding the luciferase gene (Luc+) and bearing SARS-COV-2 full-length S from indicated variants were used to infect 293T-hACE2 cells in presence of increasing concentrations of EH3 (FIG. 3C) or EH8 (FIG. 3D) at 37° C. for 1 h prior infection of 293T-ACE2 cells. FIG. 3E: Cell-to-cell fusion was measured between 293T effector cells expressing HIV-1 Tat and SARS-COV-2 S D614G which were incubated in presence of increasing concentrations of CV3-1, EH3 or EH8 at 37° C. for 1 h prior coculture with TZM-bl-hACE2 target cells. FIGS. 3C-E: Fitted curves and half maximal inhibitory antibody concentration (IC₅₀) values were determined using a normalized nonlinear regression. FIG. 3F: Cell-surface staining of CEM.NKr-Spike (Wuhan-Hu-1 strain) using increasing concentrations of CV3-1, EH3 or EH8 mAbs. Hill coefficient (h) values were determined using GraphPad Prism software. FIG. 3G: Parental CEM.NKr cells were mixed at a 1:1 ratio with CEM.NKr-Spike cells and were used as target cells. Cryopreserved PBMCs from uninfected donors were used as effector cells in a fluorescence-activated cell sorting (FACS)-based ADCC assay. The graphs shown represent the percentages of ADCC obtained in the presence of increasing concentrations of CV3-1, EH3 or EH8 mAbs. These results were obtained in at least 3 independent experiments. Error bars indicate means±SEM.

FIGS. 4A-E show the epitope mapping of RBD-specific mAbs by site-directed mutagenesis. FIGS. 4A-D: Cell-surface staining of 293T cells expressing selected full-length SARS-COV-2 S harboring RBM mutations using ACE2-Fc (FIG. 4A), CV3-1 (FIG. 4B), EH3 (FIG. 4C) and EH8 (FIG. 4D). The graphs shown represent the median fluorescence intensities (MFI) corrected for cell-surface S expression of the corresponding mutant using the CV3-25 mAb and further normalized to the MFI obtained with S D614G (WT). Dashed lines indicate the reference value obtained with S D614G (WT). Error bars indicate means±SEM. These results were obtained in at least three independent experiments. Statistical significance was tested using one-way ANOVA with a Dunnett post-test (+p <0.05; ** p <0.01; *** p <0.001; **** p <0.0001). FIG. 4E: Structural representation of SARS-COV-2 RBD depicted as a surface model (PDB: 6VW1). Amino acid substitutions able to significantly decrease the binding of indicated ligands by more than 50% compared to WT are identified.

FIG. 5 is a schematic of the EH8 CAR constructs. Two different iterations of the scFV of the CAR constructs were cloned. 1EH8 construct had the variable heavy (VH) chain sequence cloned upstream of the variable light (VL) chain sequence, and 2EH8 had the VL chain sequence upstream the VH chain sequence.

FIGS. 6A and 6B are graphs showing the results of cytotoxic assays of CAR-NK cells bearing either the 1EH8 (FIG. 6A) or 2EH8 (FIG. 6B) construct against target cells expressing the Spike protein. CAR-NK (EH8) or non-transduced NK cells (NT) were put in contact with 697 target cells expressing (697-Spike) or not (EGFP 697) the Spike protein from the original strain. Results with the 1EH8 construct having the variable heavy (VH) chain sequence cloned upstream of the variable light (VL) chain sequence are shown in FIG. 6A, while results with 2EH8 having the VL chain sequence upstream the VH chain sequence is shown in FIG. 6B. Mean of percentage of specific lysis for 3 different NK donors with standard deviation (SD) are shown. 2-Way ANOVA, Tukey's multiple comparison test, *** p<0.0001.

### DISCLOSURE OF INVENTION

The use of the terms “a” and “an” and “the” and similar referents in the context of describing the invention (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

The use of any and all examples, or exemplary language (e.g., “such as”) provided herein, is intended merely to better illustrate the invention and does not pose a limitation on the scope of the invention unless otherwise claimed.

No language in the specification should be construed as indicating any non-claimed element as essential to the practice of the invention.

Herein, the term “about” has its ordinary meaning. The term “about” is used to indicate that a value includes an inherent variation of error for the device or the method being employed to determine the value, or encompass values close to the recited values, for example within 10% or 5% of the recited values (or range of values).

As used herein the term “individual,” “patient,” or “subject” refers to individuals diagnosed with, suspected of being afflicted with, or at-risk of developing at least one disease for which the described compositions and method are useful for treating. In certain embodiments the individual is a mammal. In certain embodiments, the mammal is a mouse, rat, rabbit, dog, cat, horse, cow, sheep, pig, goat, llama, alpaca, or yak. In certain embodiments, the individual is a human.

As described herein severe SARS-COV-2 infection refers to individuals infected with SARS-COV-2 that develop difficulty breathing or persistent chest pressure or pain. Severe SARS-CoV-2 infection may require hospitalization, supplemental oxygen, and or mechanical ventilation. Many individuals are at high risk for severe SARS-COV-2 including the elderly, diabetic, or those with pre-existing cardiovascular disease.

As described herein acute respiratory distress (ARDs) refers to the fluid build-up of lung alveoli as a result of trauma or infection. ARDs is a significant life-threatening complication of many viral infections including SARS-COV-2. The antibodies and methods described herein can prevent or improve the prognosis of an individual suffering from SARS-COV-2 related ARDs.

Unless otherwise defined, all technical and scientific terms used herein have the same meaning as commonly understood by one of ordinary skill in the art to which this invention belongs.

In the studies described herein, the present inventors have identified two antibodies having the ability to neutralize SARS-COV-2 and its different variants, including the B. 1.351 variant

(South Africa) the B.1.1.7 variant (UK) as well as other variants of concern (VOC) such as P.1 and B.1.617.2, and to trigger antibody-dependent cell cytotoxicity (ADCC) in SARS-COV-2-infected cells.

Thus, in a first aspect, the present disclosure provides an antibody or an antigen binding fragment thereof comprises one of the following combinations of complementarity determining regions (CDRs):
(a) a light chain CDR1 (CDR-L1) comprising or consisting of an amino acid sequence having at least 70%, 75%, 80%, 85% or 90% identity with the sequence RASQSVSSSYLA (SEQ ID NO: 14); a CDR-L2 comprising or consisting of an amino acid sequence having at least 70%, 75%, 80%, 85% or 90% identity with the sequence GASSRAT (SEQ ID NO:17); a CDR-L3 comprising or consisting of an amino acid sequence having at least 70%, 75%, 80%, 85% or 90% identity with the sequence QQYGSSYT (SEQ ID NO:19); a heavy chain CDR1 (CDR-H1) comprising or consisting of an amino acid sequence having at least 70%, 75%, 80%, 85% or 90% identity with the sequence GITVSSN (SEQ ID NO:1); a CDR-H2 comprising or consisting of an amino acid sequence having at least 70%, 75%, 80%, 85% or 90% identity with the sequence YSGGS (SEQ ID NO:6); and a CDR-H3 comprising or consisting of an amino acid sequence having at least 70%, 75%, 80%, 85% or 90% identity with the sequence DLEMAGAFDI (SEQ ID NO: 11); or
(b) a CDR-L1 comprising or consisting of an amino acid sequence having at least 70%, 75%, 80%, 85% or 90% identity with the sequence TGTSSDVGSYNLVS (SEQ ID NO:36); a CDR-L2 comprising or consisting of an amino acid sequence having at least 70%, 75%, 80%, 85% or 90% identity with the sequence EGTKRPS (SEQ ID NO:39); a CDR-L3 comprising or consisting of an amino acid sequence having at least 70%, 75%, 80%, 85% or 90% identity with the sequence CSYAGNSTWV (SEQ ID NO:41); a CDR-H1 comprising or consisting of an amino acid sequence having at least 70%, 75%, 80%, 85% or 90% identity with the sequence GYTFSSY (SEQ ID NO:23); a CDR-H2 comprising or consisting of an amino acid sequence having at least 70%, 75%, 80%, 85% or 90% identity with the sequence SPYNGN (SEQ ID NO:28); and a CDR-H3 comprising or consisting of an amino acid sequence having at least 70%, 75%, 80%, 85% or 90% identity with the sequence DLELGGGFDY (SEQ ID NO:33).

The term “antibody or antigen-binding fragment thereof” as used herein refers to any type of antibody/antibody fragment including monoclonal antibodies (including full-length monoclonal antibodies), polyclonal antibodies, multispecific antibodies, humanized antibodies, CDR-grafted antibodies, chimeric antibodies and antibody fragments so long as they exhibit the desired antigenic specificity/binding activity. Antibody fragments comprise a portion of a full-length antibody, generally an antigen binding or variable region thereof. Examples of antibody fragments include Fab, Fab′, F(ab′)2, and Fv fragments, diabodies, linear antibodies, single-chain antibody molecules (e.g., single-chain FV, scFV), single domain antibodies (e.g., from camelids), shark

NAR single domain antibodies, and multispecific antibodies formed from antibody fragments. Antibody fragments can also refer to binding moieties comprising CDRs or antigen binding domains including, but not limited to, V_{H} regions (V_{H}, V_{H}-V_{H}), anticalins, PepBodies, antibody-T-cell epitope fusions (Troybodies) or Peptibodies.

The term “monoclonal antibody” as used herein refers to an antibody from a population of substantially homogeneous antibodies, i.e., the individual antibodies comprising the population are substantially similar and bind the same epitope(s), except for possible variants that may arise during production of the monoclonal antibody, such variants generally being present in minor amounts. Such monoclonal antibody typically includes an antibody comprising a variable region that binds a target, wherein the antibody was obtained by a process that includes the selection of the antibody from a plurality of antibodies. For example, the selection process can be the selection of a unique clone from a plurality of clones, such as a pool of hybridoma clones, phage clones or recombinant DNA clones. It should be understood that the selected antibody can be further altered, for example, to improve affinity for the target, to humanize the antibody, to improve its production in cell culture, to reduce its immunogenicity in vivo, to create a multispecific antibody, etc., and that an antibody comprising the altered variable region sequence is also a monoclonal antibody of this disclosure. In addition to their specificity, the monoclonal antibody preparations are advantageous in that they are typically uncontaminated by other immunoglobulins. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by a variety of techniques, including the hybridoma method (e.g., Kohler et al., Nature, 256:495 (1975); Harlow et al., Antibodies: A Laboratory Manual, (Cold Spring Harbor Laboratory Press, 2nd ed. 1988); Hammerling et al., in: Monoclonal Antibodies and T-Cell Hybridomas 563-681, (Elsevier, N. Y., 1981), recombinant DNA methods (see, e.g., U.S. Pat. No. 4,816,567), phage display technologies (see, e.g., Clackson et al., Nature, 352:624-628 (1991); Marks et al., J. Mol. Biol., 222:581-597 (1991); Sidhu et al., J. Mol. Biol. 338 (2): 299-310 (2004); Lee et al., J. Mol. Biol. 340 (5): 1073-1093 (2004); Fellouse, Proc. Nat. Acad. Sci. USA 101 (34): 12467-12472 (2004); and Lee et al. J. Immunol. Methods 284 (1-2): 119-132 (2004) and technologies for producing human or human-like antibodies from animals that have parts or all of the human immunoglobulin loci or genes encoding human immunoglobulin sequences (see, e.g., WO98/24893,

WO96/34096, WO96/33735, and WO91/10741, Jakobovits et al., Proc. Natl. Acad. Sci. USA, 90:2551 (1993); Jakobovits et al., Nature, 362:255-258 (1993); Bruggemann et al., Year in Immune, 7:33 (1993); U.S. Pat. Nos. 5,545,806, 5,569,825, 5,591,669 (all of GenPharm); U.S. Pat. No. 5,545,807; WO 97/17852, U.S. Pat. Nos. 5,545,807; 5,545,806; 5,569,825; 5,625,126; 5,633,425; and 5,661,016, and Marks et al., Bio/Technology, 10:779-783 (1992); Lonberg et al., Nature, 368:856-859 (1994); Morrison, Nature, 368:812-813 (1994); Fishwild et al., Nature Biotechnology, 14:845-851 (1996); Neuberger, Nature Biotechnology, 14:826 (1996); and Lonberg and Huszar, Intern. Rev. Immunol., 13:65-93 (1995).

The monoclonal antibodies herein specifically include “chimeric” or “recombinant” antibodies in which a portion of the light and/or heavy chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). Chimeric antibodies of interest herein include “humanized” antibodies.

The term “variable” refers to the fact that certain portions of the variable domains differ extensively in sequence among antibodies and are used in the binding and specificity of each particular antibody for its particular antigen. However, the variability is not evenly distributed throughout the variable domains of antibodies. It is concentrated in three segments called complementarity-determining regions (CDRs) or hypervariable regions (HVRs) both in the light-chain and heavy-chain variable domains. The more highly conserved portions of variable domains are called the framework region (FR). The variable domains of native heavy and light chains each comprise four FR regions, largely adopting a B-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies. The constant domains are not involved directly in binding an antibody to an antigen, but exhibit various effector functions, such as participation of the antibody in antibody-dependent cellular cytotoxicity (ADCC). From N-terminal to C-terminal, both light and heavy chain variable regions comprise alternating FRs and CDRs: FR1, CDR1, FR2, CDR2, FR3, CDR3 and FR4. The assignment of amino acids to each region may be made in accordance with the definitions of Kabat, Chothia (Al-Lazikani et al., J Mol Biol. 1997; 273 (4): 927-48), or IMGT (Lefranc, M.-P., Immunology Today, 18, 509 (1997)), for example.

“Fv” is the minimum antibody fragment which contains a complete antigen-recognition and binding site. In a two-chain Fv species, this region consists of a dimer of one heavy-and one light-chain variable domain in tight, non-covalent association. In a single-chain Fv species, one heavy-and one light-chain variable domain can be covalently linked by a flexible peptide linker such that the light and heavy chains can associate in a “dimeric” structure analogous to that in a two-chain Fv species. It is in this configuration that the three CDRs of each variable domain interact to define an antigen-binding site on the surface of the V_{H}-VL dimer. Collectively, the six CDRs are involved in conferring the antigen-binding specificity to the antibody. However, even a single variable domain (or half of an Fv comprising only three CDRs specific for an antigen) has the ability to recognize and bind antigen, although at a lower affinity than the entire binding site.

“Hypervariable region” or “HVR” refers to the amino acid residues of an antibody that are responsible for antigen-binding. The hypervariable region generally comprises amino acid residues from a “complementarity determining region” or “CDR” (Kabat et al., Sequences of Proteins of Immunological Interest, 5th Ed. Public Health Service, National Institutes of Health, Bethesda, Md. (1991)) and/or those residues from a “hypervariable loop” (Al-Lazikani et al., supra).

The term “complementarity determining regions” or “CDRs” when used herein refers to parts of immunological receptors that make contact with a specific ligand and determine its specificity. The CDRs of immunological receptors are the most variable part of the receptor protein, giving receptors their diversity, and are carried on six loops at the distal end of the receptor's variable domains, three loops coming from each of the two variable domains of the receptor.

As used herein, the term “framework region” refers to those portions of immunoglobulin light and heavy chain variable regions that are relatively conserved (i.e., other than the CDRs) among different immunoglobulins in a single species, as defined by Kabat et al. (supra) or Chothia (Al-Lazikani et al., supra). As used herein, a “human framework region” is a framework region that is substantially identical to the framework region of a naturally occurring human antibody.

The sequences of the CDR and FR as defined herein are defined according to the Clothia numbering scheme. However, the skilled person would understand that the amino acids forming the CDRs and FRs regions in the sequences of antibodies #3 and #8 may vary depending on the numbering scheme used. Tables 1-4 below depict the sequences of the CDRs and FRs regions of antibodies #3 and #8 according to commonly used antibody numbering schemes.

TABLE 1
Predicted FR and CDR sequences in Antibody #3 Heavy chain variable region as
determined using the abYsis tool (www.abysis.org/abysis/index.html)

| Region | Definition | Sequence Fragment (SEQ ID NO:) | Residues | Length |
|---|---|---|---|---|
| HFR1 | Chothia | EVQLVESGGGLVQPGGSLRLSCAAS----- (49) | 1-25 | 25 |
| | AbM | EVQLVESGGGLVQPGGSLRLSCAAS----- (49) | 1-25 | 25 |
| | Kabat | EVQLVESGGGLVQPGGSLRLSCAASGITVS (68) | 1-30 | 30 |
| | Contact | EVQLVESGGGLVQPGGSLRLSCAASGITV- (69) | 1-29 | 29 |
| | IMGT | EVQLVESGGGLVQPGGSLRLSCAAS----- (49) | 1-25 | 25 |
| CDR- | Chothia | GITVSSN--- (1) | 26-32 | 7 |
| H1 | AbM | GITVSSNYMT (2) | 26-35 | 10 |
| | Kabat | -----SNYMT (3) | 31-35 | 5 |
| | Contact | ----SSNYMT (4) | 30-35 | 6 |
| | IMGT | GITVSSNY-- (5) | 26-33 | 8 |
| HFR2 | Chothia | YMTWVRQAPGKGLEWVSVI (50) | 33-51 | 19 |
| | AbM | ---WVRQAPGKGLEWVS-- (70) | 36-49 | 14 |
| | Kabat | ---WVRQAPGKGLEWVS-- (71) | 36-49 | 14 |
| | Contact | ---WVRQAPGKGLE----- (72) | 36-46 | 11 |
| | IMGT | -MTWVRQAPGKGLEWVSV- (73) | 34-50 | 17 |
| CDR- | Chothia | -----YSGGS--------- (6) | 52-56 | 5 |
| H2 | AbM | ---VIYSGGSTF------- (7) | 50-58 | 9 |
| | Kabat | ---VIYSGGSTFYADSVRG (8) | 50-65 | 16 |
| | Contact | WVSVIYSGGSTF------- (9) | 47-58 | 12 |
| | IMGT | ----IYSGGST-------- (10) | 51-57 | 7 |
| HFR3 | Chothia | TFYADSVRGRFTISRDNSKNTLYLQMNSLRAEDTAVYYCAR | 57-97 | 41 |
| | | (51) | | |
| | AbM | --YADSVRGRFTISRDNSKNTLYLQMNSLRAEDTAVYYCAR | 59-97 | 39 |
| | | (73) | | |
| | Kabat | ---------RFTISRDNSKNTLYLQMNSLRAEDTAVYYCAR | 66-97 | 32 |
| | | (74) | | |
| | Contact | --YADSVRGRFTISRDNSKNTLYLQMNSLRAEDTAVYYC-- | 59-95 | 37 |
| | | (75) | | |
| | IMGT | -FYADSVRGRFTISRDNSKNTLYLQMNSLRAEDTAVYYC-- | 58-95 | 38 |
| | | (76) | | |
| CDR- | Chothia | --DLEMAGAFDI (11) | 98-107 | 10 |
| H3 | AbM | --DLEMAGAFDI (11) | 98-107 | 10 |
| | Kabat | --DLEMAGAFDI (11) | 98-107 | 10 |
| | Contact | ARDLEMAGAFD- (12) | 96-106 | 11 |
| | IMGT | ARDLEMAGAFDI (13) | 96-107 | 12 |
| HFR4 | Chothia | -WGQGTMVTVSS (52) | 108-118 | 11 |
| | AbM | -WGQGTMVTVSS (52) | 108-118 | 11 |
| | Kabat | -WGQGTMVTVSS (52) | 108-118 | 11 |
| | Contact | IWGQGTMVTVSS (77) | 107-118 | 12 |
| | IMGT | -WGQGTMVTVSS (52) | 108-118 | 11 |

TABLE 2
Predicted FR and CDR sequences in Antibody #3 Light chain variable region

| Region | Definition | Sequence Fragment (SEQ ID NO:) | Residues | Length |
|---|---|---|---|---|
| LFR1 | Chothia | EIVLTQSPGTLSLSPGERATLSC------ (45) | 1-23 | 23 |
| | AbM | EIVLTQSPGTLSLSPGERATLSC------ (45) | 1-23 | 23 |
| | Kabat | EIVLTQSPGTLSLSPGERATLSC------ (45) | 1-23 | 23 |
| | Contact | EIVLTQSPGTLSLSPGERATLSCRASQSV (61) | 1-29 | 29 |
| | IMGT | EIVLTQSPGTLSLSPGERATLSCRAS--- (62) | 1-26 | 26 |
| CDR- | Chothia | RASQSVSSSYLA-- (14) | 24-35 | 12 |
| L1 | AbM | RASQSVSSSYLA-- (14) | 24-35 | 12 |
| | Kabat | RASQSVSSSYLA-- (14) | 24-35 | 12 |
| | Contact | ------SSSYLAWY (15) | 30-37 | 8 |
| | IMGT | ---QSVSSSY---- (16) | 27-33 | 7 |
| LFR2 | Chothia | --WYQQKPGQAPRLLIY (46) | 36-50 | 15 |
| | AbM | --WYQQKPGQAPRLLIY (46) | 36-50 | 15 |
| | Kabat | --WYQQKPGQAPRLLIY (46) | 36-50 | 15 |
| | Contact | ----QQKPGQAPR---- (63) | 38-46 | 9 |
| | IMGT | LAWYQQKPGQAPRLLIY (64) | 34-50 | 17 |
| CDR- | Chothia | ----GASSRAT (17) | 51-57 | 7 |
| L2 | AbM | ----GASSRAT (17) | 51-57 | 7 |
| | Kabat | ----GASSRAT (17) | 51-57 | 7 |
| | Contact | LLIYGASSRA- (18) | 47-56 | 10 |
| | IMGT | ----GA----- | 51-52 | 2 |
| LFR3 | Chothia | -----GIPDRFSGSGSGTDFTLTISRLEPEDSAVYYC (47) | 58-89 | 32 |
| | AbM | -----GIPDRFSGSGSGTDFTLTISRLEPEDSAVYYC (47) | 58-89 | 32 |
| | Kabat | -----GIPDRFSGSGSGTDFTLTISRLEPEDSAVYYC (47) | 58-89 | 32 |
| | Contact | ----TGIPDRFSGSGSGTDFTLTISRLEPEDSAVYYC (65) | 57-89 | 33 |
| | IMGT | SSRATGIPDRFSGSGSGTDFTLTISRLEPEDSAVYYC (66) | 53-89 | 37 |
| CDR- | Chothia | QQYGSSYT (19) | 90-97 | 8 |
| L3 | AbM | QQYGSSYT (19) | 90-97 | 8 |
| | Kabat | QQYGSSYT (19) | 90-97 | 8 |
| | Contact | QQYGSSY- (20) | 90-96 | 7 |
| | IMGT | QQYGSSYT (19) | 90-97 | 8 |
| LFR4 | Chothia | -FGQGTKLEIK (48) | 98-107 | 10 |
| | AbM | -FGQGTKLEIK (48) | 98-107 | 10 |
| | Kabat | -FGQGTKLEIK (48) | 98-107 | 10 |
| | Contact | TFGQGTKLEIK (67) | 97-107 | 11 |
| | IMGT | -FGQGTKLEIK (48) | 98-107 | 10 |

TABLE 3
Predicted FR and CDR sequences in Antibody #8 Heavy chain variable region

| Region | Definition | Sequence Fragment (SEQ ID NO:) | Residues | Length |
|---|---|---|---|---|
| HFR1 | Chothia | QVQLVQSGAEVKKPGASVKVSCKAS----- (57) | 1-25 | 25 |
| | AbM | QVQLVQSGAEVKKPGASVKVSCKAS----- (57) | 1-25 | 25 |
| | Kabat | QVQLVQSGAEVKKPGASVKVSCKASGYTFS (85) | 1-30 | 30 |
| | Contact | QVQLVQSGAEVKKPGASVKVSCKASGYTF- (86) | 1-29 | 29 |
| | IMGT | QVQLVQSGAEVKKPGASVKVSCKAS----- (57) | 1-25 | 25 |
| CDR- | Chothia | GYTFSSY--- (23) | 26-32 | 7 |
| H1 | AbM | GYTFSSYGIS (24) | 26-35 | 10 |
| | Kabat | -----SYGIS (25) | 31-35 | 5 |
| | Contact | ----SSYGIS (26) | 30-35 | 6 |
| | IMGT | GYTFSSYG-- (27) | 26-33 | 8 |
| HFR2 | Chothia | GISWVRQAPGQGLEWMGWI (58) | 33-51 | 19 |
| | AbM | ---WVRQAPGQGLEWMG-- (87) | 36-49 | 14 |
| | Kabat | ---WVRQAPGQGLEWMG-- (87) | 36-49 | 14 |
| | Contact | ---WVRQAPGQGLE----- (88) | 36-46 | 11 |
| | IMGT | -ISWVRQAPGQGLEWMGW- (89) | 34-50 | 17 |
| CDR- | Chothia | -----SPYNGN--------- (28) | 52-57 | 6 |
| H2 | AbM | ---WISPYNGNTK------- (29) | 50-59 | 10 |
| | Kabat | ---WISPYNGNTKYPQKFQG (30) | 50-66 | 17 |
| | Contact | WMGWISPYNGNTK------- (31) | 47-59 | 13 |
| | IMGT | ----ISPYNGNT-------- (32) | 51-58 | 8 |
| HFR3 | Chothia | TKYPQKFQGRVTMTTDTSTNTAYMELRSLRSDDTAVYYCAR | 58-98 | 41 |
| | | (59) | | |
| | AbM | --YPQKFQGRVTMTTDTSTNTAYMELRSLRSDDTAVYYCAR | 60-98 | 39 |
| | | (90) | | |
| | Kabat | ---------RVTMTTDTSTNTAYMELRSLRSDDTAVYYCAR | 67-98 | 32 |
| | | (91) | | |
| | Contact | --YPQKFQGRVTMTTDTSTNTAYMELRSLRSDDTAVYYC-- | 60-96 | 37 |
| | | (92) | | |
| | IMGT | -KYPQKFQGRVTMTTDTSTNTAYMELRSLRSDDTAVYYC-- | 59-96 | 38 |
| | | (93) | | |
| CDR- | Chothia | --DLELGGGFDY (33) | 99-108 | 10 |
| H3 | AbM | --DLELGGGFDY (33) | 99-108 | 10 |
| | Kabat | --DLELGGGFDY (33) | 99-108 | 10 |
| | Contact | ARDLELGGGFD- (34) | 97-107 | 11 |
| | IMGT | ARDLELGGGFDY (35) | 97-108 | 12 |
| HFR4 | Chothia | -WGQGTLVTVSS (60) | 109-119 | 11 |
| | AbM | -WGQGTLVTVSS (60) | 109-119 | 11 |
| | Kabat | -WGQGTLVTVSS (60) | 109-119 | 11 |
| | Contact | YWGQGTLVTVSS (94) | 108-119 | 12 |
| | IMGT | -WGQGTLVTVSS (60) | 109-119 | 11 |

TABLE 4
Predicted FR and CDR sequences in Antibody #8 Light chain variable region

| Region | Definition | Sequence Fragment (SEQ ID NO:) | Residues | Length |
|---|---|---|---|---|
| LFR1 | Chothia | QSALTQPASVSGSPGQSITISC------ (53) | 1-22 | 22 |
| | AbM | QSALTQPASVSGSPGQSITISC------ (53) | 1-22 | 22 |
| | Kabat | QSALTQPASVSGSPGQSITISC------ (53) | 1-22 | 22 |
| | Contact | QSALTQPASVSGSPGQSITISCTGTSSD (78) | 1-28 | 28 |
| | IMGT | QSALTQPASVSGSPGQSITISCTGT--- (79) | 1-25 | 25 |
| CDR- | Chothia | TGTSSDVGSYNLVS-- (36) | 23-36 | 14 |
| L1 | AbM | TGTSSDVGSYNLVS-- (36) | 23-36 | 14 |
| | Kabat | TGTSSDVGSYNLVS-- (36) | 23-36 | 14 |
| | Contact | ------VGSYNLVSWY (37) | 29-38 | 10 |
| | IMGT | ---SSDVGSYNL---- (38) | 26-34 | 9 |
| LFR2 | Chothia | --WYQQHPDKAPKFMIY (54) | 37-51 | 15 |
| | AbM | --WYQQHPDKAPKFMIY (54) | 37-51 | 15 |
| | Kabat | --WYQQHPDKAPKFMIY (54) | 37-51 | 15 |
| | Contact | ----QQHPDKAPK---- (80) | 39-47 | 9 |
| | IMGT | VSWYQQHPDKAPKFMIY (81) | 35-51 | 17 |
| CDR- | Chothia | ----EGTKRPS (39) | 52-58 | 7 |
| | AbM | ----EGTKRPS (39) | 52-58 | 7 |
| | Kabat | ----EGTKRPS (39) | 52-58 | 7 |
| | Contact | FMIYEGTKRP- (40) | 48-57 | 10 |
| | IMGT | ----EG----- | 52-53 | 2 |
| LFR3 | Chothia | -----GVSNRFSGSKSGNTASLTISGLQAEDEADYYC (55) | 59-90 | 32 |
| | AbM | -----GVSNRFSGSKSGNTASLTISGLQAEDEADYYC (55) | 59-90 | 32 |
| | Kabat | -----GVSNRFSGSKSGNTASLTISGLQAEDEADYYC (55) | 59-90 | 32 |
| | Contact | ----SGVSNRFSGSKSGNTASLTISGLQAEDEADYYC (82) | 58-90 | 33 |
| | IMGT | TKRPSGVSNRFSGSKSGNTASLTISGLQAEDEADYYC (83) | 54-90 | 37 |
| CDR- | Chothia | CSYAGNSTWV (41) | 91-100 | 10 |
| L3 | AbM | CSYAGNSTWV (41) | 91-100 | 10 |
| | Kabat | CSYAGNSTWV (41) | 91-100 | 10 |
| | Contact | CSYAGNSTW- (42) | 91-99 | 9 |
| | IMGT | CSYAGNSTWV (41) | 91-100 | 10 |
| LFR4 | Chothia | -FGGGTKLTVL (56) | 101-110 | 10 |
| | AbM | -FGGGTKLTVL (56) | 101-110 | 10 |
| | Kabat | -FGGGTKLTVL (56) | 101-110 | 10 |
| | Contact | VFGGGTKLTVL (84) | 100-110 | 11 |
| | IMGT | -FGGGTKLTVL (56) | 101-110 | 10 |

In an embodiment, one or two residues in the above-noted CDRs sequences are substituted. In a further embodiment, one residue in the above-noted CDRs sequences is substituted.
In an embodiment, the antibody or an antigen binding fragment thereof comprises one of the following combinations of CDRs:
(a) a light chain CDR1 (CDR-L1) comprising or consisting of the sequence RASQSVSSSYLA (SEQ ID NO:14); a CDR-L2 comprising or consisting of the sequence GASSRAT (SEQ ID NO:17); a CDR-L3 comprising or consisting of the sequence QQYGSSYT (SEQ ID NO:19); a heavy chain CDR1 (CDR-H1) comprising or consisting of the sequence GITVSSN (SEQ ID NO:1); a CDR-H2 comprising or consisting of the sequence YSGGS (SEQ ID NO: 6); and a CDR-H3 comprising or consisting of the sequence DLEMAGAFDI (SEQ ID NO:11); or
(b) a CDR-L1 comprising or consisting of the sequence TGTSSDVGSYNLVS (SEQ ID NO: 36); a CDR-L2 comprising or consisting of the sequence EGTKRPS (SEQ ID NO:39); a CDR-L3 comprising or consisting of the sequence CSYAGNSTWV (SEQ ID NO:41); a CDR-H1comprising or consisting of the sequence GYTFSSY (SEQ ID NO:23); a CDR-H2 comprising or consisting of the sequence SPYNGN (SEQ ID NO:28); and a CDR-H3 comprising or consisting of the sequence DLELGGGFDY (SEQ ID NO:33).

In an embodiment, one or two residues in the above-noted CDRs sequences are substituted. In a further embodiment, one residue in the above-noted CDRs sequences are substituted.

In an embodiment, the antibody or antigen-binding fragment thereof comprises: (i) a light chain FR1 comprising or consisting of an amino acid sequence having at least 50%, 60%, 70%, 75%, 80%, 85%, 90% or 95% identity with the sequence EIVLTQSPGTLSLSPGERATLSC (SEQ ID NO: 45); (ii) a light chain FR2 comprising or consisting of an amino acid sequence having at least 50%, 60%, 70%, 75%, 80%, 85%, 90% or 95% identity with the sequence WYQQKPGQAPRLLIY (SEQ ID NO:46); (iii) a light chain FR3 comprising or consisting of an amino acid sequence having at least 50%, 60%, 70%, 75%, 80%, 85%, 90% or 95% identity with the sequence GIPDRFSGSGSGTDFTLTISRLEPEDSAVYYC (SEQ ID NO:47); (iv) a light chain FR4 comprising or consisting of an amino acid sequence having at least 50%, 60%, 70%, 75%, 80%, 85%, 90% or 95% identity with the sequence FGQGTKLEIK (SEQ ID NO:48); or (v) any combination of (i) to (iv). In an embodiment, the light chain FR1 comprises or consists of the amino acid sequence EIVLTQSPGTLSLSPGERATLSC (SEQ ID NO:45). In an embodiment, the light chain FR2 comprises or consists of the amino acid sequence WYQQKPGQAPRLLIY (SEQ ID NO: 46). In an embodiment, the light chain FR3 comprises or consists of the amino acid sequence GIPDRFSGSGSGTDFTLTISRLEPEDSAVYYC (SEQ ID NO:47). In an embodiment, the light chain FR4 comprises or consists of the amino acid sequence FGQGTKLEIK (SEQ ID NO: 48).

In an embodiment, the antibody or antigen-binding fragment thereof comprises: (i) a light chain FR1 comprising or consisting of an amino acid sequence having at least 50%, 60%, 70%, 75%, 80%, 85%, 90% or 95% identity with the sequence QSALTQPASVSGSPGQSITISC (SEQ ID NO: 53); (ii) a light chain FR2 comprising or consisting of an amino acid sequence having at least 50%, 60%, 70%, 75%, 80%, 85%, 90% or 95% identity with the sequence WYQQHPDKAPKFMIY (SEQ ID NO:54); (iii) a light chain FR3 comprising or consisting of an amino acid sequence having at least 50%, 60%, 70%, 75%, 80%, 85%, 90% or 95% identity with the sequence GVSNRFSGSKSGNTASLTISGLQAEDEADYYC (SEQ ID NO:55); (iv) a light chain FR4 comprising or consisting of an amino acid sequence having at least 50%, 60%, 70%, 75%, 80%, 85%, 90% or 95% identity with the sequence FGGGTKLTVL (SEQ ID NO:56); or (v) any combination of (i) to (iv). In an embodiment, the light chain FR1 comprises or consists of the amino acid sequence QSALTQPASVSGSPGQSITISC (SEQ ID NO:53). In an embodiment, the light chain FR2 comprises or consists of the amino acid sequence WYQQHPDKAPKFMIY (SEQ ID NO: 54). In an embodiment, the light chain FR3 comprises or consists of the amino acid sequence GVSNRFSGSKSGNTASLTISGLQAEDEADYYC (SEQ ID NO:55). In an embodiment, the light chain FR4 comprises or consists of the amino acid sequence FGGGTKLTVL (SEQ ID NO: 56).

In an embodiment, the antibody or antigen-binding fragment thereof comprises: (i) a heavy chain FR1 comprising or consisting of an amino acid sequence having at least 50%, 60%, 70%, 75%, 80%, 85%, 90% or 95% identity with the sequence EVOLVESGGGLVQPGGSLRLSCAAS (SEQ ID NO:49); (ii) a heavy chain FR2 comprising or consisting of an amino acid sequence having at least 50%, 60%, 70%, 75%, 80%, 85%, 90% or 95% identity with the sequence YMTWVRQAPGKGLEWVSVI (SEQ ID NO:50); (iii) a heavy chain FR3 comprising or consisting of an amino acid sequence having at least 50%, 60%, 70%, 75%, 80%, 85%, 90% or 95% identity with the sequence TFYADSVRGRFTISRDNSKNTLYLQMNSLRAEDTAVYYCAR (SEQ ID NO:51); (iv) a heavy chain FR4 comprising or consisting of an amino acid sequence having at least 50%, 60%, 70%, 75%, 80%, 85%, 90% or 95% identity with the sequence WGQGTMVTVSS (SEQ ID NO:52); or (v) any combination of (i) to (iv). In an embodiment, the heavy chain FR1 comprises or consists of the amino acid sequence EVOLVESGGGLVQPGGSLRLSCAAS (SEQ ID NO:49). In an embodiment, the heavy chain FR2 comprises or consists of the amino acid sequence YMTWVRQAPGKGLEWVSVI (SEQ ID NO:50). In an embodiment, the heavy chain FR3 comprises or consists of the amino acid sequence TFYADSVRGRFTISRDNSKNTLYLQMNSLRAEDTAVYYCAR (SEQ ID NO:51). In an embodiment, the heavy chain FR4 comprises or consists of the amino acid sequence WGQGTMVTVSS (SEQ ID NO:52).

In an embodiment, the antibody or antigen-binding fragment thereof comprises: (i) a heavy chain FR1 comprising or consisting of an amino acid sequence having at least 50%, 60%, 75%, or 95% with 70%, 80%, 85%, 90% identity the sequence QVQLVQSGAEVKKPGASVKVSCKAS (SEQ ID NO:57); (ii) a heavy chain FR2 comprising or consisting of an amino acid sequence having at least 50%, 60%, 70%, 75%, 80%, 85%, 90% or 95% identity with the sequence GISWVRQAPGQGLEWMGWI (SEQ ID NO:58); (iii) a heavy chain FR3 comprising or consisting of an amino acid sequence having at least 50%, 60%, 70%, 75%, 80%, 85%, 90% or 95% identity with the sequence TKYPQKFQGRVTMTTDTSTNTAYMELRSLRSDDTAVYYCAR (SEQ ID NO:59); (iv) a heavy chain FR4 comprising or consisting of an amino acid sequence having at least 50%, 60%, 70%, 75%, 80%, 85%, 90% or 95% identity with the sequence WGQGTLVTVSS (SEQ ID NO:60); or (v) any combination of (i) to (iv). In an embodiment, the heavy chain FR1 comprises or consists of the amino acid sequence QVQLVQSGAEVKKPGASVKVSCKAS (SEQ ID NO:57). In an embodiment, the heavy chain FR2 comprises or consists of the amino acid sequence GISWVRQAPGQGLEWMGWI (SEQ ID NO:58). In an embodiment, the heavy chain FR3 comprises or consists of the amino acid sequence TKYPQKFQGRVTMTTDTSTNTAYMELRSLRSDDTAVYYCAR (SEQ ID NO:59). In an embodiment, the heavy chain FR4 comprises or consists of the amino acid sequence WGQGTLVTVSS (SEQ ID NO:60).

In an embodiment, the antibody or antigen-binding fragment thereof comprises a variable light chain comprising or consisting of an amino acid sequence having at least 70%, 75%, 80%, 85%, 90% or 95% identity with the sequence

(SEQ ID NO: 22)
EIVLTQSPGTLSLSPGERATLSCRASQSVSSSYLAWYQQKPGQAPRLLI
YGASSRATGIPDRFSGSGSGTDFTLTISRLEPEDSAVYYCQQYGSSYTF
GQQTKLEIK;
or
(SEQ ID NO: 44)
QSALTQPASVSGSPGQSITISCTGTSSDVGSYNLVSWYQQHPDKAPKFM
IYEGTKRPSGVSNRFSGSKSGNTASLTISGLQAEDEADYYCCSYAGNST
WVFGGGTKLTVL.

In an embodiment, the differences relative to the reference variable light chain sequence are within one or more of the FRs underlined above. In a further embodiment, the antibody or antigen-binding fragment thereof comprises a variable light chain comprising or consisting of one of the sequences defined above.

In an embodiment, the antibody or antigen-binding fragment thereof comprises a variable heavy chain comprising or consisting of an amino acid sequence having at least 70%, 75%, 80%, 85%, 90% or 95% identity with the sequence

(SEQ ID NO: 21)
EVQLVESGGGLVQPGGSLRLSCAASGITVSSNYMTWVRQAPGKGLEWVS
VIYSGGSTFYADSVRGRFTISRDNSKNTLYLQMNSLRAEDTAVYYCARD
LEMAGAFDIWGQGTMVTVSS;
or
(SEQ ID NO: 43)
QVQLVQSGAEVKKPGASVKVSCKASGYTFSSYGISWVRQAPGQGLEWMG
WISPYNGNTKYPQKFQGRVTMTTDTSTNTAYMELRSLRSDDTAVYYCAR
DLELGGGFDYWGQGTLVTVSS.

In an embodiment, the differences relative to the reference variable heavy chain sequence are within one or more of the FRs underlined above. In a further embodiment, the antibody or antigen-binding fragment thereof comprises a variable heavy chain comprising or consisting of one of the sequences defined above.

Variations in the antibodies or antigen-binding fragments thereof described herein, can be made, for example, using any of the techniques and guidelines for conservative and non- conservative mutations set forth, for instance, in U.S. Pat. No. 5,364,934. Variations may be a substitution, deletion or insertion of one or more codons encoding the antibody that results in a change in the amino acid sequence as compared with the native sequence antibody. Optionally the variation is by substitution of at least one amino acid with any other amino acid in one or more of the domains of the anti-NTSR1 antibody or antigen-binding fragment thereof. Guidance in determining which amino acid residue may be inserted, substituted or deleted without adversely affecting the desired activity may be found by comparing the sequence of the antibody or antigen-binding fragment thereof with that of homologous known protein molecules and minimizing the number of amino acid sequence changes made in regions of high homology. Amino acid substitutions can be the result of replacing one amino acid with another amino acid having similar structural and/or chemical properties, such as the replacement of a leucine with a serine, i.e., conservative amino acid replacements. Insertions or deletions may optionally be in the range of about 1 to 5 amino acids. The variation allowed may be determined by systematically making insertions, deletions or substitutions of amino acids in the sequence and testing the resulting variants for activity exhibited by the full-length or mature native sequence. In embodiment, the variant exhibit at least 50%, 55% or 60%, preferably at least 65, 70, 75, 80, 90, 95, 96, 97, 98 or 99% sequence identity with the sequence of the antibody or antigen-binding fragment thereof described herein, and maintain the ability to specifically bind to SARS-COV-2 Spike protein.

“Identity” refers to sequence identity between two polypeptides. Percent (%) sequence identity with respect to a reference polypeptide sequence is the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are known for instance, using publicly available computer software such as BLAST, BLAST-2, ALIGN or Megalign (DNASTAR) software. Appropriate parameters for aligning sequences are able to be determined, including algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows: 100 times the fraction X/Y, where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

Covalent modifications of antibodies or antigen-binding fragments thereof are included within the scope of this disclosure. Covalent modifications include reacting targeted amino acid residues of the antibody or antigen-binding fragment thereof with an organic derivatizing agent that is capable of reacting with selected side chains or the N- or C-terminal residues of the antibody or antigen-binding fragment thereof. Other modifications include deamidation of glutaminyl and asparaginyl residues to the corresponding glutamyl and aspartyl residues, respectively, hydroxylation of proline and lysine, phosphorylation of hydroxyl groups of seryl or threonyl residues, methylation of the α-amino groups of lysine, arginine, and histidine side chains (T.E. Creighton, Proteins: Structure and Molecular Properties, W.H. Freeman & Co., San Francisco, pp. 79-86 (1983)), acetylation of the N-terminal amine, and amidation of any C-terminal carboxyl group.

Other types of covalent modification of the antibody or antigen-binding fragment thereof included within the scope of this disclosure include altering the native glycosylation pattern of the antibody or antigen-binding fragment thereof (Beck et al., Curr. Pharm. Biotechnol. 9:482-501, 2008; Walsh, Drug Discov. Today 15:773-780, 2010), and linking the antibody or antigen-binding fragment thereof to one of a variety of nonproteinaceous polymers, e.g., polyethylene glycol (PEG), polypropylene glycol, or polyoxyalkylenes, in the manner set forth in U.S. Pat. Nos. 4,640,835; 4,496,689; 4,301,144; 4,670,417; 4,791,192 or 4,179,337.

The antibody or antigen-binding fragment thereof may further comprise one or more modifications that confer additional biological properties to antibody or antigen-binding fragment thereof such as protease resistance, plasma protein binding, increased plasma half-life, intracellular penetration, etc. Such modifications include, for example, covalent attachment of molecules/moiety to the antibody or antigen-binding fragment thereof such as fatty acids (e.g., C₆-C₁₈), attachment of proteins such as albumin (see, e.g., U.S. Pat. No. 7,268,113); sugars/polysaccharides (glycosylation), biotinylation or PEGylation (see, e.g., U.S. Pat. Nos. 7,256,258 and 6,528,485). The above description of modification of the antibody or antigen-binding fragment thereof does not limit the scope of the approaches nor the possible modifications that can be engineered. Thus, in another aspect, the present disclosure provides a conjugate comprising the antibody or antigen-binding fragment thereof described herein and one or more additional molecules or agents (hereinafter secondary molecules or agents). The antibody or antigen-binding fragment thereof may be conjugated to any type of synthetic or natural secondary molecules or agents, such as peptides. proteins, saccharides/polysaccharides, lipids, naturally-occurring or synthetic polymers/co-polymers, etc. to modify one or more properties of the antibody or antigen-binding fragment thereof.

In an embodiment, the conjugate comprises a covalent link or bond between the antibody or antigen-binding fragment thereof and the molecule conjugated thereto. The molecule may be conjugated directly to the antibody or antigen-binding fragment thereof, or indirectly via a linker. The linker may be a polypeptide linker comprising one or more amino acids or another type of chemical linker (e.g., a carbohydrate linker, a lipid linker, a fatty acid linker, a polyether linker, PEG, etc.

In another embodiment, the molecule may be conjugated/attached to the side chain of one the amino acids of the antibody or antigen-binding fragment thereof. Methods for conjugating moieties to side-chains of amino acids are well known in the art. For example, chemical groups that react with primary amines (—NH₂) present in the side-chain of lysine residues such as isothiocyanates, isocyanates, acyl azides, NHS esters, sulfonyl chlorides, aldehydes, glyoxals, epoxides, oxiranes, carbonates, aryl halides, imidoesters, carbodiimides, anhydrides, and fluorophenyl esters may be used to conjugate the molecule to the antibody or antigen-binding fragment thereof. Most of these groups conjugate to amines by either acylation or alkylation. Cysteine residues present in the self-assembling domain may also be used to attach the antigen.

In an embodiment, the antibody or antigen-binding fragment thereof is labelled or conjugated with one or more moieties. The antibody or antigen-binding fragment thereof may be labeled with one or more labels such as a biotin label, a fluorescent label, an enzyme label, a coenzyme label, a chemiluminescent label, or a radioactive isotope label. In an embodiment, the antibody or antigen-binding fragment thereof is labelled with a detectable label, for example a fluorescent moiety (fluorophore). Useful detectable labels include fluorescent compounds (e.g., fluorescein isothiocyanate, Texas red, rhodamine, fluorescein, Alexa Fluor® dyes, and the like), radiolabels, enzymes (e.g., horseradish peroxidase, alkaline phosphatase and others commonly used in a protein detection assays), streptavidin/biotin, and colorimetric labels such as colloidal gold, colored glass or plastic beads (e.g., polystyrene, polypropylene, latex, etc.). Chemiluminescent compounds may also be used. Such labelled antibodies or antigen-binding fragments thereof may be useful, for example, for the detection of SARS-COV-2 and/or SARS-CoV-2-infected cells in vivo or in vitro, e.g., by flow cytometry, immunohistochemistry, etc. The antibody or antigen-binding fragment thereof can also be conjugated to detectable or affinity tags that facilitate detection and/or purification of the antibody or antigen-binding fragment thereof. Such tags are well known in the art. Examples of detectable or affinity tags include polyhistidine tags (His-tags), polyarginine tags, polyaspartate tags, polycysteine tags, polyphenylalanine tags, glutathione S-transferase (GST) tags, Maltose binding protein (MBP) tags, calmodulin binding peptide (CBP) tags, Streptavidin/Biotin-based tags, HaloTag®, Profinity exact® tags, epitope tags (such as FLAG, hemagglutinin (HA), HSV, S/S1, c-myc, KT3, T7, V5, E2, and Glu-Glu epitope tags), reporter tags such as β-galactosidase (β-gal), alkaline phosphatase (AP), chloramphenicol acetyl transferase (CAT), and horseradish peroxidase (HRP) tags (see, e.g., Kimple et al., Curr Protoc Protein Sci. 2013; 73: Unit-9.9).

In certain embodiment, the antibody or antigen-binding fragment thereof (e.g., scFV) is comprised with a chimeric antigen receptor (CAR). A CAR typically comprises a ligand-binding domain (e.g., an antibody or antibody fragment such as a single-chain variable fragment (scFv) as described herein) that provides specificity for the desired antigen (e.g., the Spike protein) linked to an activating intracellular domain portion, such as a T cell or NK cell activating domain, providing a primary activation signal, in some aspects via linkers and/or transmembrane domain(s). In particular embodiments, the CAR comprises an intracellular signaling domain, which includes an activating cytoplasmic signaling domain (also interchangeably called an intracellular signaling region), such as an activating cytoplasmic (intracellular) domain capable of inducing a primary activation signal in an immune cell (T cell, NK cell, for example), a cytoplasmic signaling domain of a T cell receptor (TCR) component (e.g. a cytoplasmic signaling domain of a CD3-zeta (CD3 ζ) chain or a functional variant or signaling portion thereof) and/or that comprises an immunoreceptor tyrosine-based activation motif (ITAM).

In some embodiments, the CAR further includes a spacer, which may be or include at least a portion of an immunoglobulin constant region or variant or modified version thereof, such as a hinge region, e.g., an IgG4 hinge region, and/or a CH1/CL and/or Fc region. In some embodiments, the constant region or portion is of a human IgG, such as IgG4 or IgG1. In some aspects, the portion of the constant region serves as a spacer region between the antigen-recognition component, e.g., scFv, and transmembrane domain. The spacer can be of a length that provides for increased responsiveness of the cell following antigen binding, as compared to in the absence of the spacer. Exemplary spacers include those having at least about 10 to 220 amino acids, about 10 to 200 amino acids, about 10 to 175 amino acids, about 10 to 150 amino acids, about 10 to 125 amino acids, about 10 to 100 amino acids, about 10 to 75 amino acids, about 10 to 50 amino acids, about 10 to 40 amino acids, about 10 to 30 amino acids, about 10 to 20 amino acids, or about 10 to 15 amino acids, and including any integer between the endpoints of any of the listed ranges. Exemplary spacers include IgG4 hinge alone, IgG4 hinge linked to CH2 and CH3 domains, or IgG4 hinge linked to the CH3 domain. Exemplary spacers include, but are not limited to, those described in Hudecek et al. (2013) Clin. Cancer Res., 19:3153 or PCT patent publication number WO 2014/031687.

The antigen/ligand recognition domain (e.g., an antibody or antibody fragment such as a single-chain variable fragment (scFv) as described herein) is generally is linked to one or more intracellular signaling components, such as signaling components that mimic activation through an antigen receptor complex, such as a TCR or NK receptor complex, in the case of a CAR, and/or signal via another cell surface receptor. Thus, in some embodiments, the antigen-binding component (e.g., antibody or antibody fragment as described herein) is linked to one or more transmembrane and intracellular signaling domains. In some embodiments, the transmembrane domain is fused to the extracellular domain. In some instances, the transmembrane domain is selected or modified by amino acid substitution to avoid binding of such domains to the transmembrane domains of the same or different surface membrane proteins to minimize interactions with other members of the receptor complex.

The transmembrane domain in some embodiments is derived either from a natural or from a synthetic source. Where the source is natural, the domain in some aspects is derived from any membrane-bound or transmembrane protein. Transmembrane regions include those derived from (i.e., comprise at least the transmembrane region(s) of) the alpha, beta or zeta chain of the TCR, CD28, CD3 epsilon, CD45, CD4, CD5, CD8, CD9, CD16, CD22, CD33, CD37, CD64, CD80, CD86, CD134, CD137, CD154. Alternatively, the transmembrane domain in some embodiments is synthetic. In some aspects, the synthetic transmembrane domain comprises predominantly hydrophobic residues such as leucine and valine. In some aspects, a triplet of phenylalanine, tryptophan and valine will be found at each end of a synthetic transmembrane domain.

Among the intracellular signaling domains are those that mimic or approximate a signal through a natural antigen receptor, a signal through such a receptor in combination with a costimulatory receptor, and/or a signal through a costimulatory receptor alone. In some embodiments, a short oligo-or polypeptide linker, for example, a linker of between 2 and 10 amino acids in length, such as one comprising glycines and serines, e.g., glycine-serine doublet, is present and forms a linkage between the transmembrane domain and the cytoplasmic signaling domain of the CAR.

The receptor, e.g., the CAR, generally includes at least one intracellular signaling component or components. In some embodiments, the receptor includes an intracellular component of a TCR complex, such as a TCR CD3 chain that mediates T-cell activation and cytotoxicity, e.g., CD3 ζ chain. Thus, in some aspects, the CAR is linked to one or more cell signaling modules. In some embodiments, cell signaling modules include CD3 transmembrane domain, CD3 intracellular signaling domains, and/or other CD transmembrane domains. In some embodiments, the receptor, e.g., CAR, further includes a portion of one or more additional molecules such as Fc receptor γ, CD8, CD4, CD25, or CD16. In some aspects, the CAR includes a primary cytoplasmic signaling sequence that regulates primary activation of the TCR complex. Primary cytoplasmic signaling sequences that act in a stimulatory manner may comprise signaling motifs which are known as immunoreceptor tyrosine-based activation motifs or ITAMs. Examples of ITAM comprising primary cytoplasmic signaling sequences include those derived from TCR or CD3 ζ, FcR gamma or FcR beta. In some embodiments, cytoplasmic signaling molecule(s) in the CAR comprise(s) a cytoplasmic signaling domain, portion thereof, or sequence derived from CD3 Zζin some embodiments, to promote full activation, a component for generating a secondary or co-stimulatory signal is also included in the CAR, such as the signaling domain of a costimulatory receptor such as CD28, 4-1BB, OX40, DAP10, and ICOS. In some aspects, an additional CAR is expressed in the same cell and provides the component for generating the secondary or costimulatory signal. In some cases, CARs are referred to as first, second, and/or third generation CARs. In some aspects, a first-generation CAR is one that solely provides an antigen-receptor (e.g., CD3-chain) induced signal upon antigen binding; in some aspects, a second-generation CARs is one that provides such a signal and costimulatory signal, such as one including an intracellular signaling domain from a costimulatory receptor such as CD28 or CD137; in some aspects, a third generation CAR in some aspects is one that includes multiple costimulatory domains of different costimulatory receptors.

In an embodiment, the present disclosure provides a cell expressing the CAR ad described herein. In an embodiment, the cell is an immune cell, such as a T cell or a NK cell.

A further aspect of the present disclosure provides nucleic acids encoding the antibody or antigen-binding fragment described herein. The isolated nucleic acid may be a synthetic DNA, a non-naturally occurring mRNA, or a cDNA, for example. The nucleic acid may be inserted within a plasmid, vector, or transcription or expression cassette. The nucleic acids encoding the antibody or antigen-binding fragment described herein may be made and the expressed antibodies or antigen-binding fragments described may be tested using conventional techniques well known in the art.

In another aspect, the present disclosure provides a cell, for example a recombinant host cell, expressing the antibody or antigen-binding fragment described herein. Methods of preparing antibodies or antigen-binding fragments comprise expressing the encoding nucleic acid(s) in a host cell under conditions to produce the antibodies or antigen-binding fragments, and recovering the antibodies or antigen-binding fragments. The process of recovering the antibodies or antigen-binding fragments may comprise isolation and/or purification of the antibodies or antigen-binding fragments. The method of production may comprise formulating the antibodies or antigen-binding fragments into a composition including at least one additional component, such as a pharmaceutically acceptable excipient.

The term “recombinant host cell” (or simply “host cell”), as used herein, is intended to refer to a cell into which exogenous DNA has been introduced. It should be understood that such terms are intended to refer not only to the particular subject cell, but, to the progeny of such a cell. Because certain modifications may occur in succeeding generations due to either mutation or environmental influences, such progeny may not, in fact, be identical to the parent cell, but are still included within the scope of the term “host cell” as used herein. Preferably host cells include prokaryotic and eukaryotic cells selected from any of the Kingdoms of life. To produce the antibody or antigen-binding fragment thereof recombinantly, the nucleic acid or nucleic acids encoding the light and heavy chains of the antibody or antigen-binding fragment thereof are introduced in a cell which is able to produce the recombinant antibody. Examples thereof include CHO-K1 (ATCC CCL-61), DUKXB11 (ATCC CCL-9096), Pro-5 (ATCC CCL-1781), CHO-S (Life Technologies®, Cat #11619), rat myeloma cell YB2/3HL.P2. G11.16Ag.20 (also called YB2/0), mouse myeloma cell NSO, mouse myeloma cell SP2/0-Ag14 (ATCC No. CRL1581), mouse P3-X63-Ag8653 cell (ATCC No. CRL1580), CHO cell in which a dihydrofolate reductase gene is defective, lectin resistance-acquired Lec13, CHO cell in which a1,6-fucosyltransaferse gene is defective, rat YB2/3HL.P2. G11.16Ag.20 cell (ATCC No. CRL1662), CHO-3E7 cells (expressing a truncated but functional form of EBNA1, U.S. Pat. No. 8,637,315) or the like. After introduction of the expression vector, transformants which stably express a recombinant antibody are selected by culturing them in a medium for animal cell culture containing an agent such as G418 sulfate or the like. Examples of the medium for animal cell culture include RPMI1640 medium (manufactured by Invitrogen®), GIT medium (manufactured by Nihon Pharmaceutical®), EX-CELL301® medium (manufactured by JRH®), IMDM medium (manufactured by Invitrogen®), Hybridoma-SFM medium (manufactured by Invitrogen®), media obtained by adding various additives such as FBS to these media, or the like. The recombinant antibody can be produced and accumulated in a culture supernatant by culturing the obtained transformants in a medium. The expression level and antigen binding activity of the recombinant antibody in the culture supernatant can be measured by ELISA or the like. Also, in the transformant, the expression level of the recombinant antibody can be increased by using DHFR amplification system or the like. The recombinant antibody can be purified from the culture supernatant of the transformant by using a protein A column. In addition, the recombinant antibody can be purified by combining the protein purification methods such as gel filtration, ion-exchange chromatography, ultrafiltration or the like. The molecular weight of the H chain or the L chain of the purified recombinant antibody or the antibody molecule as a whole is determined by polyacrylamide gel electrophoresis, Western blotting, or the like.

Suitable vectors comprising nucleic acid(s) encoding the antibody or antigen-binding fragment described herein can be chosen or constructed, containing appropriate regulatory sequences, including promoter sequences, terminator sequences, polyadenylation sequences, enhancer sequences, marker genes and other sequences as appropriate. Vectors may be plasmids, phage, phagemids, adenoviral, AAV, lentiviral, for example. Techniques and protocols for manipulation of nucleic acid, for example in preparation of nucleic acid constructs, mutagenesis, sequencing, introduction of DNA into cells, and gene expression, are well known in the art.

The term “vector”, as used herein, is intended to refer to a nucleic acid molecule capable of transporting another nucleic acid to which it has been linked. One type of vector is a “plasmid”, which refers to a circular double stranded DNA loop into which additional DNA segments may be ligated. Another type of vector is a viral vector, wherein additional DNA segments may be ligated into the viral genome.

Certain vectors are capable of autonomous replication in a host cell into which they are introduced (e.g., bacterial vectors having a bacterial origin of replication and episomal mammalian vectors). Other vectors (e.g., non-episomal mammalian vectors) can be integrated into the genome of a host cell upon introduction into the host cell, and thereby are replicated along with the host genome. Moreover, certain vectors are capable of directing the expression of genes to which they are operatively linked. Such vectors are referred to herein as “recombinant expression vectors” (or simply, “expression vectors”). In general, expression vectors of utility in recombinant DNA techniques are often in the form of plasmids. In the present specification, “plasmid” and “vector” may be used interchangeably as the plasmid is the most commonly used form of vector. However, the disclosure is intended to include such other forms of expression vectors, such as viral vectors (e.g., replication defective retroviruses, adenoviruses and adeno-associated viruses), which serve equivalent functions.

Introducing such nucleic acids into a host cell can be accomplished using techniques well known in the art. For eukaryotic cells, suitable techniques may include calcium phosphate transfection, DEAE-Dextran, electroporation, liposome-mediated transfection, and transduction using retroviruses or other viruses, for example. For bacterial cells, suitable techniques may include calcium chloride transformation, electroporation, and transfection using bacteriophage. The introduction may be followed by causing or allowing expression from the nucleic acid, e.g. by culturing host cells under conditions for expression of the gene. In one embodiment, the nucleic acid of the invention is integrated into the genome, e.g., chromosome, of the host cell. Integration may be promoted by inclusion of sequences which promote recombination with the genome, in accordance with standard techniques.

### Compositions Comprising the Antibodies or Antigen-Binding Fragments Thereof

In another aspect, the present disclosure provides a composition comprising the antibody or antigen-binding fragment thereof defined herein. In an embodiment, the composition further comprises the above-mentioned antibody or an antigen-binding fragment thereof and a carrier or excipient, in a further embodiment a pharmaceutically acceptable carrier or excipient. Such compositions may be prepared in a manner well known in the pharmaceutical art by mixing the antibody or an antigen-binding fragment thereof having a suitable degree of purity with one or more optional pharmaceutically acceptable carriers or excipients (see Remington: The Science and Practice of Pharmacy, by Loyd V Allen, Jr, 2012, 22^{nd} edition, Pharmaceutical Press; Handbook of Pharmaceutical Excipients, by Rowe et al., 2012, 7^{th} edition, Pharmaceutical Press). The carrier/excipient can be suitable for administration of the antibody or an antigen-binding fragment thereof by any conventional administration route, for example, for oral, intravenous, parenteral, subcutaneous, intramuscular, intracranial, intraorbital, ophthalmic, intraventricular, intracapsular, intraspinal, intrathecal, epidural, intracisternal, intraperitoneal, intranasal or pulmonary (e.g., aerosol) administration. In an embodiment, the carrier/excipient is adapted for administration of the antibody or an antigen-binding fragment thereof by the intravenous or subcutaneous route. In an embodiment, the carriers/excipients are adapted for administration of the antibody or an antigen-binding fragment thereof by the intravenous route. In another embodiment, the carriers/excipients are adapted for administration of the antibody or an antigen-binding fragment thereof by the subcutaneous route.

An “excipient” as used herein has its normal meaning in the art and is any ingredient that is not an active ingredient (drug) itself. Excipients include for example binders, lubricants, diluents, fillers, thickening agents, disintegrants, plasticizers, coatings, barrier layer formulations, lubricants, stabilizing agent, release-delaying agents and other components. “Pharmaceutically acceptable excipient” as used herein refers to any excipient that does not interfere with effectiveness of the biological activity of the active ingredients and that is not toxic to the subject, i.e., is a type of excipient and/or is for use in an amount which is not toxic to the subject. Excipients are well known in the art, and the present system is not limited in these respects. In certain embodiments, one or more formulations of the dosage form include excipients, including for example and without limitation, one or more binders (binding agents), thickening agents, surfactants, diluents, release-delaying agents, colorants, flavoring agents, fillers, disintegrants/dissolution promoting agents, lubricants, plasticizers, silica flow conditioners, glidants, anti-caking agents, anti-tacking agents, stabilizing agents, anti-static agents, swelling agents and any combinations thereof. As those of skill would recognize, a single excipient can fulfill more than two functions at once, e.g., can act as both a binding agent and a thickening agent. As those of skill will also recognize, these terms are not necessarily mutually exclusive. Examples of commonly used excipient include water, saline, phosphate buffered saline, dextrose, glycerol, ethanol, and the like, as well as combinations thereof. In many cases, it will be preferable to include isotonic agents, for example, sugars, polyalcohols, such as mannitol, sorbitol, or sodium chloride in the composition. Additional examples of pharmaceutically acceptable substances are wetting agents or auxiliary substances, such as emulsifying agents, preservatives, or buffers, which increase the shelf life or effectiveness. In an embodiment, the antibody or antigen-binding fragment thereof defined herein is encapsulated in a vesicle or vesicle-like particle, such as a lipid vesicle (e.g., liposome).

The composition may also comprise one or more additional active agents for the treatment the targeted disease/condition or for the management of symptom(s) of the targeted disease/condition (e.g., pain killers, anti-nausea agents, anti-inflammatory agents, immunotherapeutic agents, etc.).

The antibody or antigen-binding fragment thereof described herein may comprise one or more excipients to make the antibody or antigen-binding fragment thereof suitable for nasal or oral administration. The antibody or antigen-binding fragment thereof described herein may comprise one or more excipients to make them suitable for oral administration (e.g., nebulization). Such formulations allow delivery of the antibody or antigen-binding fragment thereof to specific sites of action along the nasopharyngeal, trachea, and/or lungs.

In certain embodiments, described herein, is a method of delivery of a composition comprising an antibody comprising any one or more of the CDRs or VH/VLs regions of antibody #3 to the respiratory system of an individual infected with SARS-COV-2, the method comprising administering a nebulized antibody formulation comprising an antibody comprising any one or more of the CDRs or VH/VL regions of antibody #3 to the individual.

In certain embodiments, described herein, is the use of a nebulized antibody or antigen-binding fragment formulation comprising any one or more of the CDRs or VH/VLs regions of antibody #3 for delivering the antibody or antigen-binding fragment to the respiratory system of an individual infected with SARS-COV-2.

In certain embodiments, described herein, is a method of delivery of a composition comprising an antibody comprising any one or more of the CDR or VH/VL regions of antibody #8 to the respiratory system of an individual infected with SARS-COV-2, the method comprising administering a nebulized antibody formulation comprising an antibody comprising any one or more of the CDRs or VH/VL regions of antibody #8 to the individual.

In certain embodiments, described herein, is the use of a nebulized antibody or antigen-binding fragment formulation comprising any one or more of the CDRs or VH/VLs regions of antibody #8 for delivering the antibody or antigen-binding fragment to the respiratory system of an individual infected with SARS-COV-2.

In certain embodiments, described herein, is a method of treating a SARS-COV-2 infection in an individual comprising administering to the individual an antibody composition comprising an antibody comprising one or more CDRs or VH/VL regions of antibody #3 to the respiratory system of the individual by nebulization.

In certain embodiments, described herein, is the use of a nebulized antibody or antigen-binding fragment formulation comprising any one or more of the CDRs or VH/VLs regions of antibody #3 for treating a SARS-COV-2 infection in an individual, wherein the formulation is for delivery of the antibody or antigen-binding fragment to the respiratory system of the individual.

In certain embodiments, described herein, is a method of treating a SARS-COV-2 infection in an individual comprising administering to the individual an antibody composition comprising an antibody comprising one or more CDRs or VH/VL regions of antibody #8 to the respiratory system of the individual by nebulization.

In certain embodiments, described herein, is the use of a nebulized antibody or antigen-binding fragment formulation comprising any one or more of the CDRs or VH/VLs regions of antibody #8 for treating a SARS-COV-2 infection in an individual, wherein the formulation is for delivery of the antibody or antigen-binding fragment to the respiratory system of the individual.

In certain embodiments, described herein, is a method of treating ARD associated with a SARS-COV-2 infection in an individual comprising administering to the individual an antibody composition comprising an antibody comprising one or more CDRs or VH/VL regions of antibody #3 to the respiratory system of the individual by nebulization.

In certain embodiments, described herein, is the use of a nebulized antibody or antigen-binding fragment formulation comprising any one or more of the CDRs or VH/VLs regions of antibody #3 for treating ARD associated with a SARS-COV-2 infection in an individual, wherein the formulation is for delivery of the antibody or antigen-binding fragment to the respiratory system of the individual.

In certain embodiments, described herein, is a method of treating ARD associated with a SARS-COV-2 infection in an individual comprising administering to the individual an antibody composition comprising an antibody comprising one or more CDRs or VH/VL regions of antibody #8 to the respiratory system of the individual by nebulization.

In certain embodiments, described herein, is the use of a nebulized antibody or antigen-binding fragment formulation comprising any one or more of the CDRs or VH/VLs regions of antibody #8 for treating ARD associated with a SARS-COV-2 infection in an individual, wherein the formulation is for delivery of the antibody or antigen-binding fragment to the respiratory system of the individual.

Antibodies and antigen-binding fragments thereof described herein may be nebulized using any suitable means such as a jet nebulizer (i.e., atomizer), a soft-mist inhaler, an ultrasonic wave nebulizer, or a vibrating mesh nebulizer.

### Uses of the Antibodies or Antigen-Binding Fragments Thereof

The present disclosure also provides methods and uses of the antibody, antigen-binding fragment thereof, or pharmaceutical composition described herein for the prevention and/or treatment of SARS-COV-2 infection and/or associated diseases and symptoms.

In another aspect, the present disclosure provides a method for preventing a SARS-COV-2 infection or a related disease (Coronavirus disease 2019, COVID-19), in a subject in need thereof, the method comprising administering to the subject an effective amount of the antibody, antigen-binding fragment thereof, or pharmaceutical composition described herein. The present disclosure also provides the use of antibody, antigen-binding fragment thereof, or pharmaceutical composition described herein for preventing SARS-COV-2 infection or a related disease (e.g., COVID-19) in a subject. The present disclosure also provides the use of the antibody, antigen-binding fragment thereof, or pharmaceutical composition described herein for the manufacture of a medicament for preventing SARS-COV-2 infection or a related disease (e.g., COVID-19) in a subject.

In another aspect, the present disclosure provides a method for preventing a SARS-COV-2 infection or a related disease (Coronavirus disease 2019, COVID-19), in a subject in need thereof, the method comprising administering to the subject an effective amount of a cell expressing a chimeric antigen receptor comprising the antibody or antigen-binding fragment thereof described herein. The present disclosure also provides the use of a cell expressing a chimeric antigen receptor comprising the antibody or antigen-binding fragment thereof described herein for preventing SARS-COV-2 infection or a related disease (e.g., COVID-19) in a subject. The present disclosure also provides the use of a cell expressing a chimeric antigen receptor comprising the antibody or antigen-binding fragment thereof described herein for the manufacture of a medicament for preventing SARS-COV-2 infection or a related disease (e.g., COVID-19) in a subject.

In another aspect, the present disclosure provides a method for reducing the risk of developing COVID-19, or the severity of COVID-19, in a subject in need thereof, the method comprising administering to the subject an effective amount of the antibody, antigen-binding fragment thereof, or pharmaceutical composition described herein. The present disclosure also provides the use of the antibody, antigen-binding fragment thereof, or pharmaceutical composition described herein for reducing the risk of developing COVID-19, or the severity of COVID-19, in a subject. The present disclosure also provides the antibody, antigen-binding fragment thereof, or pharmaceutical composition described herein for use in reducing the risk of developing COVID-19, or the severity of COVID-19, in a subject.

In another aspect, the present disclosure provides a method for reducing the risk of developing COVID-19, or the severity of COVID-19, in a subject in need thereof, the method comprising administering to the subject an effective amount of a cell expressing a chimeric antigen receptor comprising the antibody or antigen-binding fragment thereof described herein. The present disclosure also provides the use of a cell expressing a chimeric antigen receptor comprising the antibody or antigen-binding fragment thereof described herein for reducing the risk of developing COVID-19, or the severity of COVID-19, in a subject. The present disclosure also provides a cell expressing a chimeric antigen receptor comprising the antibody or antigen-binding fragment thereof described herein for use in reducing the risk of developing COVID-19, or the severity of COVID-19, in a subject.

In another aspect, the present disclosure provides a method (in vitro or in vivo) for blocking the entry of SARS-COV-2 in a cell, such as an ACE2-expressing cell, comprising contacting the cell and/or virus with an effective amount of the antibody or antigen-binding fragment thereof described herein. The present disclosure provides the use of the antibody or antigen-binding fragment thereof described herein for blocking the entry of SARS-COV-2 in a cell, such as an ACE2-expressing cell. The present disclosure provides the use of the antibody or antigen-binding fragment thereof described herein for the manufacture of a medicament for blocking the entry of SARS-COV-2 in a cell, such as an ACE2-expressing cell. The present disclosure provides the antibody or antigen-binding fragment thereof described herein for use in blocking the entry of SARS-COV-2 in a cell, such as an ACE2-expressing cell.

In another aspect, the present disclosure provides a method (in vitro or in vivo) for blocking the entry of SARS-COV-2 in a cell, such as an ACE2-expressing cell, comprising contacting the cell and/or virus with an effective amount of a cell expressing a chimeric antigen receptor comprising the antibody or antigen-binding fragment thereof described herein. The present disclosure provides the use of a cell expressing a chimeric antigen receptor comprising the antibody or antigen-binding fragment thereof described herein for blocking the entry of SARS-CoV-2 in a cell, such as an ACE2-expressing cell. The present disclosure provides the use of a cell expressing a chimeric antigen receptor comprising the antibody or antigen-binding fragment thereof described herein for the manufacture of a medicament for blocking the entry of SARS-CoV-2 in a cell, such as an ACE2-expressing cell. The present disclosure provides a cell expressing a chimeric antigen receptor comprising the antibody or antigen-binding fragment thereof described herein for use in blocking the entry of SARS-COV-2 in a cell, such as an ACE2-expressing cell.

In an embodiment, the methods and uses defined herein are for the prevention, treatment and/or management of infections by the Wuhan original SARS-COV-2 strain. In another embodiment, the methods and uses defined herein are for the prevention, treatment and/or management of infections by variants of the Wuhan original SARS-COV-2 strain, such as the B.1.1.7 (also known as VOC-202012/01), 501Y.V2 (B.1.351), P.1 (B.1.1.28.1), or B.1.617.2 (delta) variant, as well as other variants of concern (VOC) such as B.1.429, B.1.526, B.1.525, and A.23.1 (see, e.g., www.cdc.gov/coronavirus/2019-ncov.cases-updates/variant-surveillance/variant-info.html).

For the prevention, treatment or reduction in the severity of a given disease or condition (viral disease such as COVID-19), the appropriate dosage of the cell, antibody, antigen-binding fragment thereof, or pharmaceutical composition described herein will depend on the type of disease or condition to be treated, the severity and course of the disease or condition, whether the cell, antibody, antigen-binding fragment thereof, or pharmaceutical composition is administered for preventive or therapeutic purposes, previous therapy, the patient's clinical history and response to the antibody, antigen-binding fragment thereof, or pharmaceutical composition, and the discretion of the attending physician. The antibody, antigen-binding fragment thereof, or pharmaceutical composition described herein may be suitably administered to the patient at one time or over a series of treatments. Preferably, it is desirable to determine the dose-response curve in vitro, and then in useful animal models prior to testing in humans. The present disclosure provides dosages for the antibody or antigen-binding fragment thereof, or pharmaceutical composition. For example, depending on the type and severity of the disease, about 1 μg/kg to to 1000 mg per kg (mg/kg) of body weight per day. Further, the effective dose may be 0.5 mg/kg, 1 mg/kg, 5 mg/kg, 10 mg/kg, 15 mg/kg, 20 mg/kg/25 mg/kg, 30 mg/kg, 35 mg/kg, 40 mg/kg, 45 mg/kg, 50 mg/kg, 55 mg/kg, 60 mg/kg, 70 mg/kg, 75 mg/kg, 80 mg/kg, 90 mg/kg, 100 mg/kg, 125 mg/kg, 150 mg/kg, 175 mg/kg, 200 mg/kg, and may increase by 25 mg/kg increments up to 1000 mg/kg, or may range between any two of the foregoing values. A typical daily dosage might range from about 1 μg/kg to 100 mg/kg or more, depending on the factors mentioned above. For repeated administrations over several days or longer, depending on the condition, the treatment is sustained until a desired suppression of disease symptoms occurs. However, other dosage regimens may be useful. The progress of this therapy is easily monitored by conventional techniques and assays.

As used herein the term “treating” or “treatment” in reference to viral infection or disease is meant to refer to administration of the agent after infection that leads to a reduction/improvement in one or more symptoms or pathological features associated with said viral disease (COVID-19). Non-limiting examples include a decrease in viral load, reduction of cough, fever, fatigue, shortness of breath, reduction/prevention of acute respiratory distress syndrome (ARDS), reduction/prevention of multi-organ failure, septic shock, and blood clots, hospitalization, etc.

As used herein the term “preventing” or “prevention” in reference to viral infection or disease is meant to refer to administration of the agent prior to infection that leads to protection from being infected or from developing the viral disease (e.g., COVID-19), to a delay in the development of the disease, or to a reduction of one or more symptoms or pathological features associated with the viral disease.

In an embodiment, the administration/use of the cell, antibody, antigen-binding fragment thereof, or pharmaceutical composition described herein delays the onset of one or more symptoms of SARS-COV-2-caused infection (e.g., COVID-19).

The cell, antibody, antigen-binding fragment thereof, or pharmaceutical composition described herein may be used alone or in combination with other prophylactic agents such as anti-virals, anti-inflammatory agents, vaccines, immunotherapies, etc. The combination of active agents and/or compositions comprising same may be administered or co-administered (e.g., consecutively, simultaneously, at different times) in any conventional dosage form. Co-administration in the context of the present disclosure refers to the administration of more than one therapeutic in the course of a coordinated treatment to achieve an improved clinical outcome. Such co-administration may also be coextensive, that is, occurring during overlapping periods of time. For example, a first agent (e.g., the cell, antibody or antigen-binding fragment thereof described herein) may be administered to a patient before, concomitantly, before and after, or after a second active agent (e.g., an antiviral or anti-inflammatory agent) is administered. The agents may in an embodiment be combined/formulated in a single composition and thus administered at the same time. In another embodiment, the cell, antibody or antigen-binding fragment thereof described herein is used in combination with one or more additional anti-SARS-CoV-2 antibodies. In a further embodiment, the cell, antibody or antigen-binding fragment thereof described herein and the one or more additional anti-SARS-COV-2 antibodies are present in the same composition, e.g., in an antibody cocktail.

In an embodiment, the cell, antibody or antigen-binding fragment thereof is administered/used prior to exposure to SARS-COV-2. In another embodiment, the cell, antibody or antigen-binding fragment thereof is administered/used after exposure to SARS-COV-2. In another embodiment, the cell, antibody or antigen-binding fragment thereof is administered/used prior to and after exposure to SARS-COV-2.

In an embodiment, the cell, antibody or antigen-binding fragment thereof is administered/used prior to development of COVID-19. In another embodiment, the cell, antibody or antigen-binding fragment thereof is administered/used after development of COVID-19. In another embodiment, the cell, antibody or antigen-binding fragment thereof is administered/used prior to and after development of COVID-19.

In another aspect, provided herein is a method of detecting the presence of SARS-COV-2 in a sample by contacting the sample with an antibody or antigen-binding fragment thereof of the disclosure, and detecting the presence or absence of an antibody-antigen complex, thereby detecting the presence of a SARS-COV-2 in a sample. Any suitable sample can be used in the methods of the disclosure. In some embodiments, the sample can be obtained from blood, cheek scraping or swab, nasal swab, saliva, biopsy, urine, feces, sputum, nasal aspiration, or semen. In some embodiments, the sample is obtained from blood. In some embodiments, the sample is saliva, blood, plasma, or serum. In some embodiments, the sample can be a sample collected from a surface suspected of being contaminated with SARS-COV-2. In an embodiment, the antibody or antigen-binding fragment thereof is bound to a detectable label such as a fluorophore, a radioactive label, a colloidal gold particle, a magnetic particle, a quantum dot, etc.

As used herein, the term “subject” is taken to mean warm blooded animals such as mammals, for example, cats, dogs, mice, guinea pigs, horses, bovine cows, sheep and humans. In an embodiment, the subject is a mammal, and more particularly a human.

In an embodiment, the subject or patient has a weakened immune system and a reduced ability to fight infections and other diseases. In an embodiment, the subject or patient is an elderly subject or patient. In another embodiment, the subject or patient is an immunodepressed or immunocompromised subject or patient.

### MODE(S) FOR CARRYING OUT THE INVENTION

The present invention is illustrated in further details by the following non-limiting examples.

### Example 1: Materials and Methods

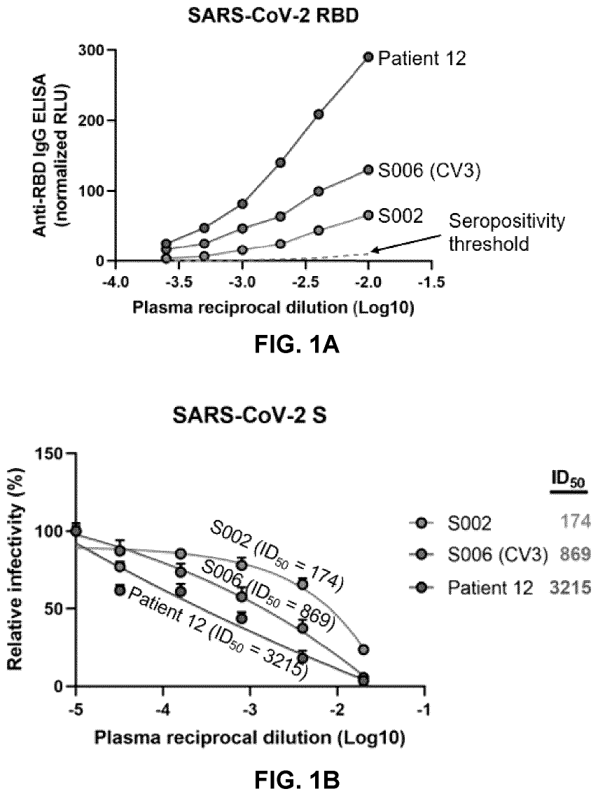

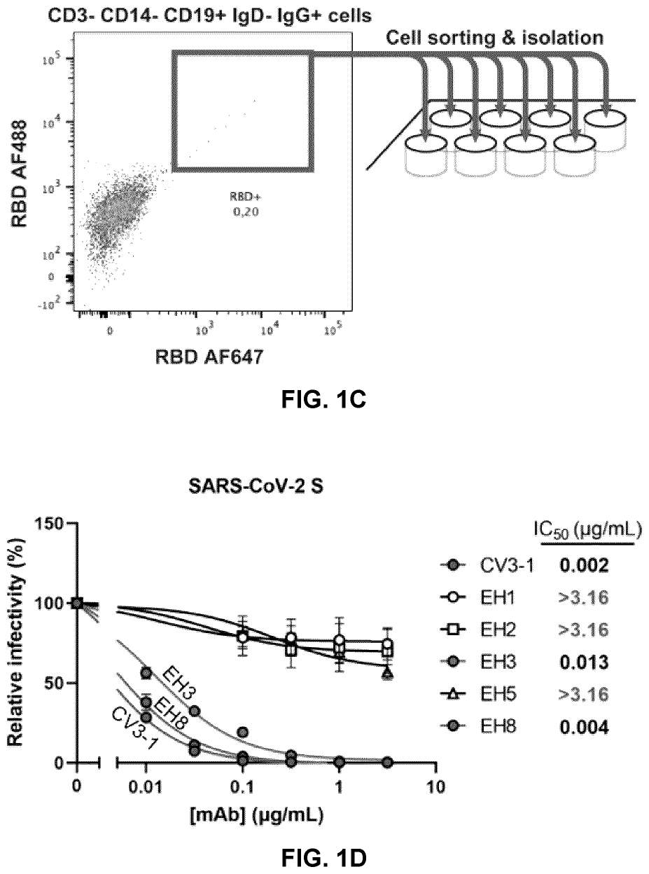

Patients. Patients presenting a Multisystem inflammatory syndrome in children (MIS-C) were recruited in a clinical research protocol after providing a written informed consent form (IRB CHU Sainte-Justine). Plasma from these patients were analysed for the presence of antibodies against SARS-COV-2 using a previously described ELISA assay. This assay detects antibodies (IgM, IgA, IgG) against the SARS-COV-2 S glycoprotein receptor binding domain (RBD) with 100% specificity^{9.11.12}. Exceptionally high titers of anti-RBD antibodies were found in one particular patient (FIGS. 1A-B), and characterisation of his antibody response was further pursued. This plasma sample was then tested for its capacity to block viral entry using a well-established neutralization assay based on a lentiviral pseudotyping neutralization assay (with SARS-COV-2 S)^{9.11-14}. Specificity was evaluated using viral particles pseudotyped with VSV-G glycoprotein and SARS-COV-1 glycoproteins. Finally, cross-reactivity using a flow-based assay expressing common (hCoV-229E-S, hCoV-NL63-S, hCoV-OC43) and pathogenic (hCoV1-S, hCoV2-S) full-length S glycoproteins were also performed.

Isolation of anti-RBD secreting cells. PBMCs from this patient had been harvested and isolated through a ficoll-Paque™ gradient. The objective of isolating the B cell clones that were secreting a specific anti-SARS-COV-2 antibody with a high neutralization capacity was pursued. For this purpose, two million (2×10⁶) isolated PBMCs were labelled with the following antibodies for flow cytometry sorting: RBD-AlexaFluor™647 (homemade), RBD-AlexaFluor™ 488 (homemade), anti-hCD19-PE (clone HIB19, Biolegend), anti-hlgG-BV786 (clone G18-145, BD Biosciences), anti-hCD3-BV395 (clone SK7, BD Biosciences), anti-hCD14-PE-Cy7 (clone M5E2, BD Biosciences), anti-hlgD-BV650 (clone IA6-2, Biolegend) and 7-AAD (BD Biosciences) as a viability marker. Gating strategy is depicted in FIG. 1C. A total of 9 RBD⁺CD19⁺IgG⁺CD3^{neg} CD14^{neg} IgD^{neg}7AAD^{neg} cells were sorted as single cells (1 cell per well). A library of cDNA was produced for each of those 9 single cells in order to identify the BCR sequence.

Library generation. Cells were sorted in 96 well plates in lysis buffer and reverse transcription and cDNA pre-amplification were performed as described¹⁵, performing 23 cycles of pre-amplification. cDNA quality was assessed on a BioAnalyzer (Agilent) using a high-sensitivity chip. V (D) J amplification was performed in two steps as described in the V (D) J library preparation kit from 10× Genomics (PN-1000016) according to the manufacturer's instructions and adding to the first amplification the enrichment primer 1 and the enrichment primer 2 to the second amplification step (see Table for the sequences of the enrichment primers). These primers are designed to bind to the template switching oligo used in the cDNA generation and add to the amplicon the sequence of the Illumina read1 primer. 5 ng of cDNA was used as input to the target amplification and 11 PCR cycles were performed for the first enrichment and 13 cycles for the second enrichment. After SPRI cleanup, the targeted amplification result was assessed on a BioAnalyser.

Following amplification of the V (D) J regions from cDNA, library construction was performed following the V (D) J reagents kit from 10× Genomics (PN-1000016) protocol for fragmentation, end repair, A-tailing, adaptor ligation and index PCR. The resulting libraries were sequenced on both an Illumina Nextera and an Oxford nanopore instrument.

TABLE 5
Sequences of the enrichment primers

| | |
|---|---|
| Enrichment Fw1 TSO binding D6 | CTA CAC GAC GCT CTT CCG ATC T AG |
| | CAGT ggt atc aac gca (SEQ ID NO: 95) |
| Enrichment Fw2 TSO binding D6 | CTA CAC GAC GCT CTT CCG ATC T AGC |
| | (SEQ ID NO: 96) |

BCR sequencing and assembly. Amplicons were submitted to Oxford Nanopore sequencing using the native barcoding (EXP-NBD104, Oxford Nanopore Technologies) and ligation sequencing protocol (SQK-sequencing data was base-called and demultiplexed with Guppy version 4.0.14 using configuration file dna_r9.4.1_450bps_hac.cfg. Demultiplexed reads were filtered and assembled into draft contigs using MAFFT, which were then polished using 4successive rounds of Minimap2 and RACON using parameters “--secondary=no” and “-m 8-x-6-g-8”, respectively. The resulting contigs were then subjected to Medaka consensus correction with default parameters. A final round of consensus polishing was performed with Illumina short reads using Nextpolish with default parameters. IgBLAST was used to assess the sequence identity of the assembled BCRs, the CDR3 sequence and the V (D) J genes.

Cloning anti-RBD antibody from identified sequences & antibody production. From the 9 different anti-RBD specific B cells isolated from the patient, 7 were successfully sequenced. Two sequences were identical, leading to a total of 6 different sequences. These assembled BCR sequences were synthesized into GeneBlocks (IDT DNA) and then cloned in the pTRIOZ-hlgG1 plasmid (InVivogen) using the restriction enzyme SgrAI (NEB) and BsiWI-HF (NEB) for the variable domain of light chain, as well as Agel-HF (NEB) and Nhel-HF (NEB) for the variable domain of heavy chain. A sanger sequencing has been performed to ensure the quality of the cloned sequences. Using these vectors, anti-RBD antibodies were then produced, purified and validated.

Flow cytometry analysis of cell-surface staining. 293T human embryonic kidney cells (obtained from ATCC) were maintained at 37° C. under 5% CO₂ in Dulbecco's modified Eagle's medium (DMEM) (Wisent) containing 5% fetal bovine serum (FBS) (VWR) and 100 μg/ml of penicillin-streptomycin (Wisent). The SARS-COV-2 Spike expressor was reported previously (Hoffmann M, Kleine-Weber H, Schroeder S, Kruger N, Herrler T, Erichsen S, Schiergens T S, Herrler G, Wu N H, Nitsche A, Muller M A, Drosten C, Pohlmann S. 2020. SARS-COV-2 Cell Entry Depends on ACE2 and TMPRSS2 and Is Blocked by a Clinically Proven Protease Inhibitor. Cell 181:271-280 e8). SARS-COV-2 Spike mutations were introduced using the QuikChange II XL site-directed mutagenesis protocol (Stratagene). The presence of the desired mutations was determined by automated DNA sequencing. The plasmid encoding the Spike of the B.1.1.7 variant was codon-optimized and synthesized by Genscript. Using the standard calcium phosphate method, 10 μg of Spike expressor and 2 μg of a green fluorescent protein (GFP) expressor (pIRES2-eGFP) was transfected into 2×10⁶ 293T cells. At 48 hours post transfection, 293T cells were stained with the monoclonal antibodies (mAb) at a final concentration of 5 μg/mL. Alexa Fluor™647-conjugated goat anti-human IgG (H+L) Abs (Invitrogen) were used as secondary antibodies. The percentage of transfected cells (GFP+ cells) was determined by gating the living cell population based on the basis of viability dye staining (Aqua Vivid, Invitrogen). Samples were acquired on a LSRII cytometer (BD Biosciences) and data analysis was performed using Flow Jo v10.7.1 (Tree Star).

Neutralization assay. Target cells were infected with single-round luciferase-expressing lentiviral particles. Briefly, 293T cells were transfected by the calcium phosphate method with the pNL4.3 R-E-Luc plasmid (NIH AIDS Reagent Program) and a plasmid encoding for SARS-COV-2 Spike at a ratio of 5:4. Two days post-transfection, cell supernatants were harvested and stored at −80° C. until use. 293T-ACE2 target cells (9) were seeded at a density of 1×10⁴ cells/well in 96-well luminometer-compatible tissue culture plates (Perkin Elmer) 24 h before infection. Recombinant viruses in a final volume of 100 pL were incubated with the indicated semi-log diluted antibody concentrations for 1 h at 37° C. and were then added to the target cells followed by incubation for 48 h at 37° C.; cells were lysed by the addition of 30 μL of passive lysis buffer (Promega) followed by one freeze-thaw cycle. An LB941 TriStar luminometer (Berthold Technologies) was used to measure the luciferase activity of each well after the addition of 100 μL of luciferin buffer (15 mM MgSO₄, 15 mM KPO₄ [pH 7.8], 1 mM ATP, and 1 mM dithiothreitol) and 50 μL of 1 mM d-luciferin potassium salt.

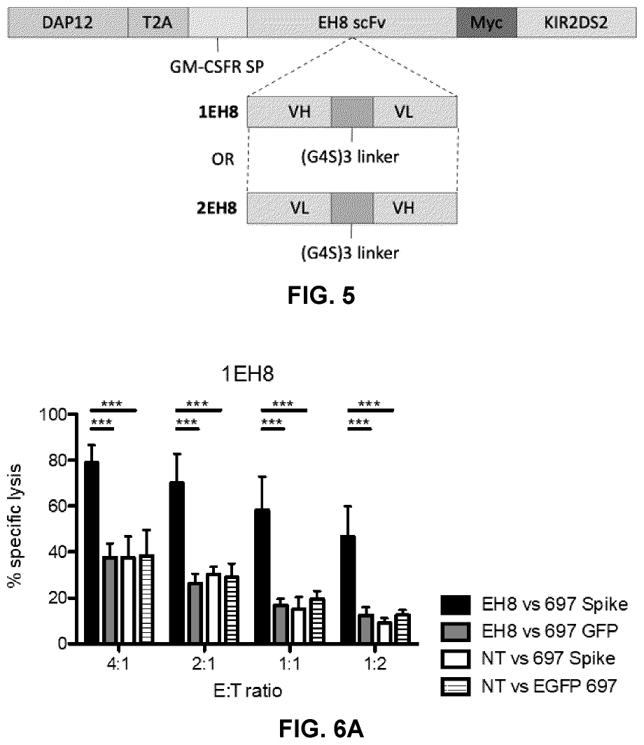

Generation of a Chimeric Antigen Receptor (CAR) using the sequence of antibody #8. The single chain variable fragment (scFv) of the CAR constructs were generated based on the sequence of antibody #8. Two iterations of the scFv were created (1EH8 and 2EH8), by alternating the order of the light and heavy chains (FIG. 5). 1EH8 construct had the variable heavy (V_{H}) chain sequence cloned upstream of the variable light (VL) chain sequence, while 2EH8 had the VL chain sequence cloned upstream of the V_{H} chain sequence. The aim was to create CAR-NK cells, henceforth a CAR architecture based on the signaling domains of KIR2DS2 transmembrane domain and co-stimulatory molecule DAP12¹⁶ was chosen. CAR plasmids were packaged into baboon envelope pseudotyped lentiviral vectors (BaEV), allowing for primary NK cell transduction¹⁷. Primary NK cells were isolated from blood samples of healthy donors and amplified using the NK activation and expansion system (NKAES) based on genetically modified K562 feeder cell lines expressing membrane-bound IL-21, together with 4-1BB ligand¹⁸. NKAES cells were then transduced with a lentivirus containing the anti-spike EH8 CAR construct and expanded. After 1 week, cells were sorted for CD56⁺CD3⁻Myc⁺ expressing cells and re-expanded.

### Example 2: Identification and Characterization of Neutralizing anti-SARS-COV-2 Antibodies

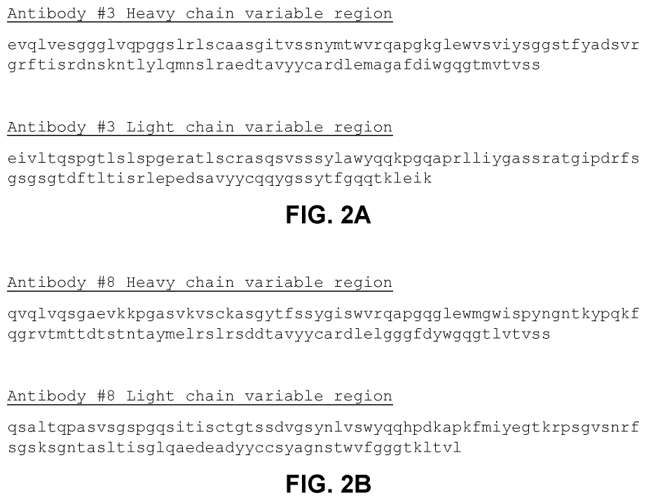

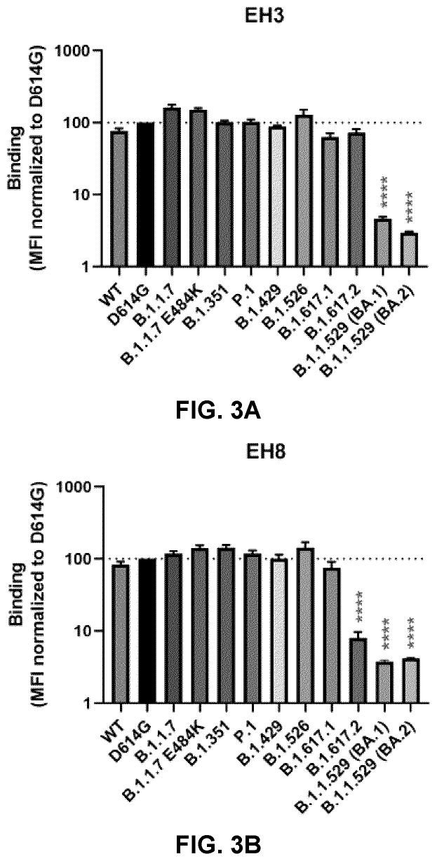

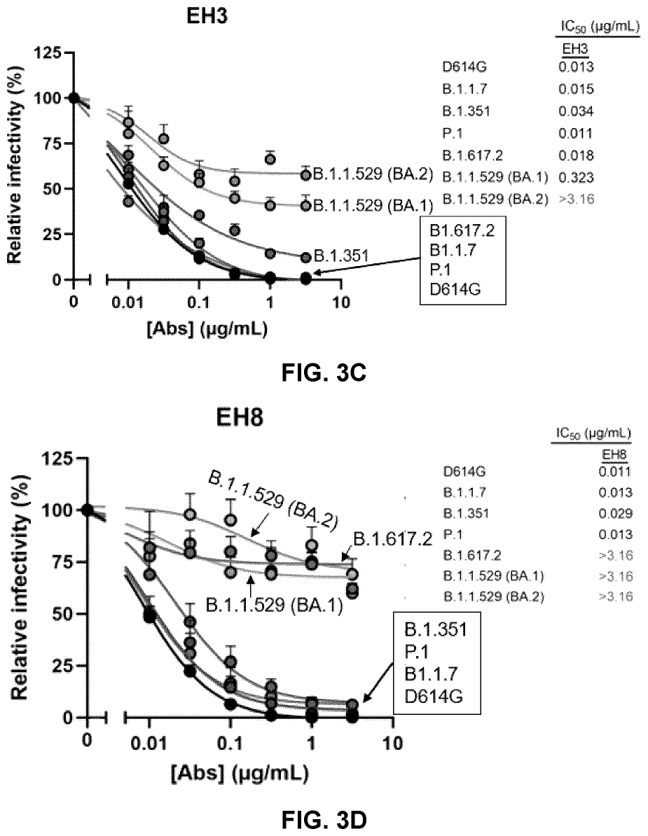

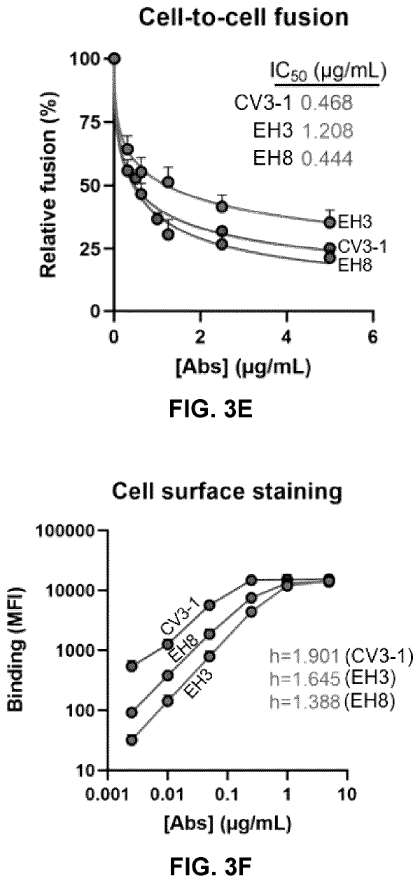

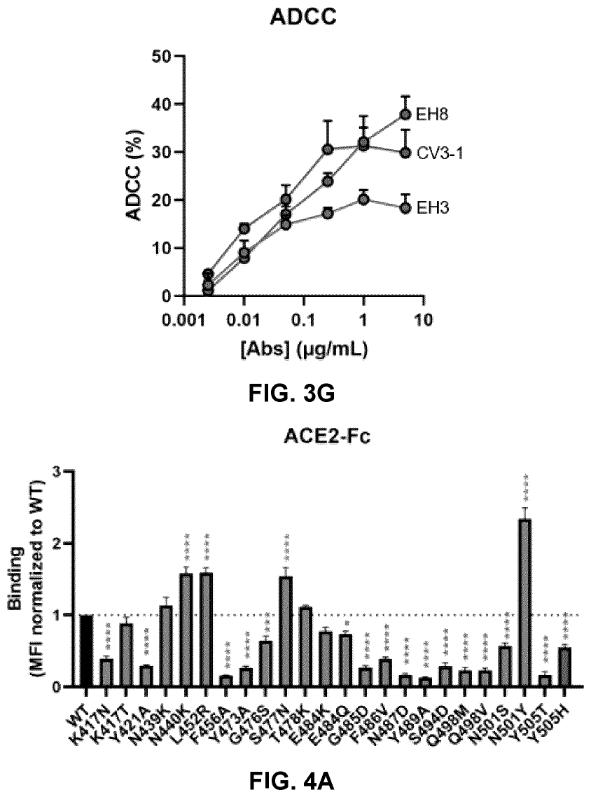

Among the six antibodies cloned and produced, only two were shown to have the ability to recognize and neutralize different SARS-COV-2 Spike variants and to induce Antibody-dependent cell-mediated cytotoxicity (ADCC). These antibodies are referred herein as antibody #3 (or EH3) and antibody #8 (EH8), and the amino acid sequences of the variable light and heavy chains are depicted in FIG. 2A (antibody #3) and FIG. 2B (antibody #8). The results depicted in FIGS. 3A-B show that antibody #3 (EH3) is able to recognize full-length Spike harboring mutations from different SARS-COV-2 variants expressed at the cell surface of 293T cells expressing, namely mutations from the B.1.1.7 variant, the B. 1.351 variant, the P.1 variant, the B1.429 variant, the B.1.526 variant, the B.1.617.1 variant and the B. 1.617.2 variant.

The results depicted in FIGS. 3C and 3D show that antibody #3 (EH3, FIG. 3C) and antibody #8 (EH8, FIG. 3D) are able to block the infection of 293T-ACE2 cells by pseudoviral particles bearing the Spike glycoprotein harboring mutations from different SARS-COV-2 variants.

The results depicted in FIG. 3E show that antibodies #3 and #8 have the ability to inhibit cell-to-cell fusion between 293T effector cells expressing HIV-1 Tat and SARS-COV-2 S D614G and TZM-bl-hACE2 target cells.

FIG. 3F shows that antibodies #3 and #8 have the ability to stain CEM.NKr-Spike cells (Wuhan-Hu-1 strain)

These results provide evidence that antibody #3 and antibody #8 have the ability to bind to SARS-COV-2 S proteins from several variants and to neutralize infection of ACE2-expressing cell by SARS-COV-2.

It was next assessed whether antibody #3 and antibody #8 were able to induce antibody-dependent cell cytotoxicity (ADCC) against cells expressing the SARS-COV-2 Spike protein. The results depicted in FIG. 3G show that antibody #3 and antibody #8 have the ability to induce ADCC in CEM.NKr cells expressing SARS-COV-2 Spike protein in a dose-dependent manner. These results provide evidence that antibody #3 and antibody #8 have the ability to bind to cells infected by SARS-COV-2 and to trigger ADCC.

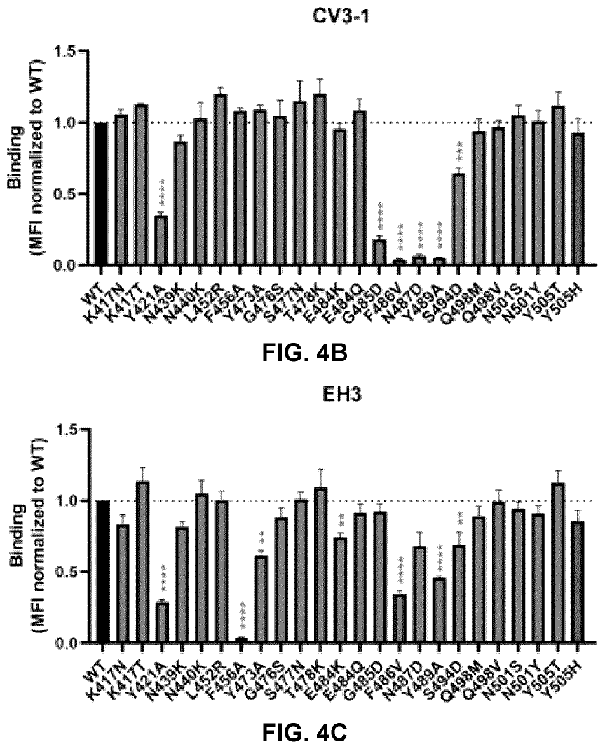

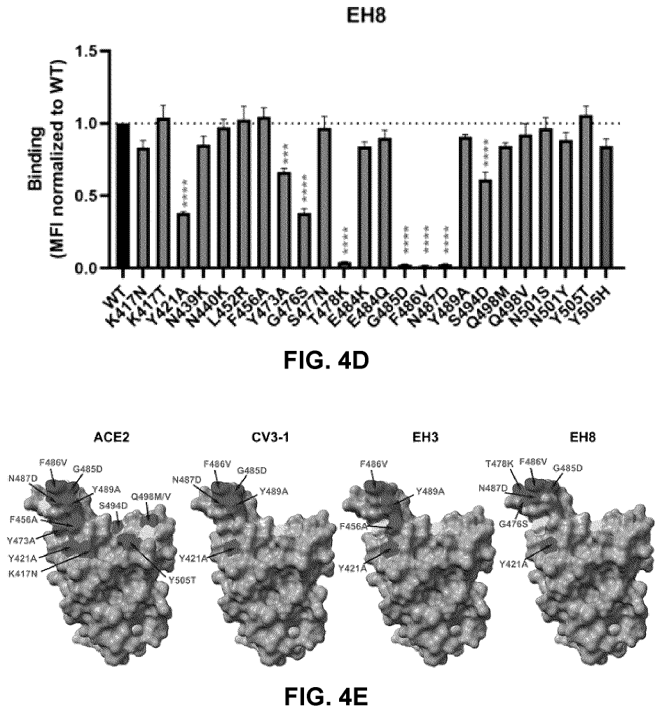

In order to identify the epitope recognized by antibody #3 and antibody #8 on the S protein, cell-surface staining of 293T cells expressing selected full-length SARS-COV-2 S harboring RBM mutations was performed. As shown in FIG. 4C, binding of antibody #3 to SARS-CoV-2 S protein is reduced or abrogated by the following amino acid substitutions: Y421A, F456A, Y473A, E484K, F486V, Y489A and S494D. The following amino acid substitutions were found to reduce or abrogate the binding of antibody #8 to SARS-COV-2 S protein: Y421A, Y473A, G476S, T478K, G485D, F486V, N487D and S494D (FIG. 4D). Based on these results, a structural representation of the SARS-COV-2 RBD with the putative epitope recognized by antibody #3 and antibody #8 was generated (FIG. 4E).

### Example 3: Characterization of the Chimeric Antigen Receptor (CAR) Comprising the Sequence of Antibody #8

To assess the efficacy and specificity of the anti-Spike CAR EH8 constructs, cytotoxic assays against target cells (697 cell line) expressing spike-GFP fusion protein (697-Spike) or not expressing spike (697-GFP) were conducted. CAR-NK cells and non-transduced NK cells were respectively plated over top of the target cells at effector: target (E: T) ratios of 4:1, 2:1, 1:1 and 1:2, leaving a control well containing only target cells. Plates were incubated for 24 hours at 37° C. and 5% CO₂. Upon incubation, 7AAD was added and samples were assessed by flow cytometry. Specific lysis was then calculated as published¹⁷.

EH8 CAR NK cells showed specific cytotoxicity against spike-expressing targets. Indeed,

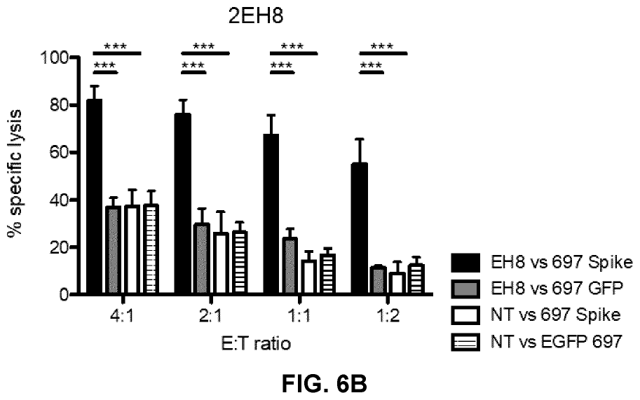

EH8 CAR NK cells showed significantly more lysis of spike-expressing targets than spike non-expressing targets and were more effective than non-transduced NK cells (FIGS. 6A-B) while non-transduced (NT) NK cells did not preferentially display cytotoxicity against spike-expressing targets (p<0.0001, 2-way ANOVA). These results were reproducible among different donors (n=3). Both conformation of EH8, 1EH8 (FIG. 6A) or 2EH8 (FIG. 6B) performed similarly.

Altogether, these results suggest that the variable sequence of antibody #8 can be used to design CARs.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context 5clearly dictates otherwise. List of sequences described herein

| SEQ ID | | | |
|---|---|---|---|
| NO: | Sequence | Description | Nomenclature |
| 1 | GITVSSN | #3 HCDR1 | Chothia |
| 2 | GITVSSNYMT | #3 HCDR1 | AbM |
| 3 | SNYMT | #3 HCDR1 | Kabat |
| 4 | SSNYMT | #3 HCDR1 | Contact |
| 5 | GITVSSNY | #3 HCDR1 | IMGT |
| 6 | YSGGS | #3 HCDR2 | Chothia |
| 7 | VIYSGGSTF | #3 HCDR2 | AbM |
| 8 | VIYSGGSTFYADSVRG | #3 HCDR2 | Kabat |
| 9 | WVSVIYSGGSTF | #3 HCDR2 | Contact |
| 10 | IYSGGST | #3 HCDR2 | IMGT |
| 11 | DLEMAGAFDI | #3 HCDR3 | Chothia, AbM, |
| | | | Kabat |
| 12 | ARDLEMAGAFD | #3 HCDR3 | Contact |
| 13 | ARDLEMAGAFDI | #3 HCDR3 | IMGT |
| 14 | RASQSVSSSYLA-- | #3 LCDR1 | Chothia, AbM, |
| | | | Kabat |
| 15 | ------SSSYLAWY | #3 LCDR1 | Contact |
| 16 | ---QSVSSSY---- | #3 LCDR1 | IMGT |
| 17 | ----GASSRAT | #3 LCDR2 | Chothia, AbM, |
| | | | Kabat |
| 18 | LLIYGASSRA- | #3 LCDR2 | Contact |
| -- | ----GA----- | #3 LCDR2 | IMGT |
| 19 | QQYGSSYT | #3 LCDR3 | Chothia, AbM, |
| | | | Kabat, IMGT |
| 20 | QQYGSSY- | #3 LCDR3 | Contact |
| 21 | evqlvesggglvqpggslrlscaasgitvssnymtwvrqapgkgle | #3 VH | |
| | wvsviysggstfyadsvrgrftisrdnskntlylqmnslraedtavyyc | | |
| | ardlemagafdiwgqgtmvtvss | | |
| 22 | eivltqspgtlslspgeratlscrasqsvsssylawyqqkpgqaprlliy | #3 VL | |
| | gassratgipdrfsgsgsgtdftltisrlepedsavyycqqygssytfgq | | |
| | qtkleik | | |
| 23 | GYTFSSY--- | #8 HCDR1 | Chothia |
| 24 | GYTFSSYGIS | #8 HCDR1 | AbM |
| 25 | -----SYGIS | #8 HCDR1 | Kabat |
| 26 | ----SSYGIS | #8 HCDR1 | Contact |
| 27 | GYTFSSYG-- | #8 HCDR1 | IMGT |
| 28 | ---------SPYNGN--------- | #8 HCDR2 | Chothia |
| 29 | -------WISPYNGNTK------- | #8 HCDR2 | AbM |
| 30 | -------WISPYNGNTKYPQKFQG | #8 HCDR2 | Kabat |
| 31 | ----WMGWISPYNGNTK------- | #8 HCDR2 | Contact |
| 32 | --------ISPYNGNT-------- | #8 HCDR2 | IMGT |
| 33 | ---DLELGGGFDY | #8 HCDR3 | Chothia, AbM, |
| | | | Kabat |
| 34 | -ARDLELGGGFD- | #8 HCDR3 | Contact |
| 35 | -ARDLELGGGFDY | #8 HCDR3 | IMGT |
| 36 | TGTSSDVGSYNLVS-- | #8 LCDR1 | Chothia, AbM, |
| | | | Kabat |
| 37 | ------VGSYNLVSWY | #8 LCDR1 | Contact |
| 38 | ---SSDVGSYNL---- | #8 LCDR1 | IMGT |
| 39 | -------EGTKRPS----- | #8 LCDR2 | Chothia, AbM, |
| | | | Kabat |
| 40 | FMIYEGTKRP- | #8 LCDR2 | Contact |
| -- | -------EG----- | #8 LCDR2 | IMGT |
| 41 | CSYAGNSTWV | #8 LCDR3 | Chothia, AbM, |
| | | | Kabat, IMGT |
| 42 | CSYAGNSTW- | #8 LCDR3 | Contact |
| 43 | qvqlvqsgaevkkpgasvkvsckasgytfssygiswvrqapgqgle | #8 VH | |
| | wmgwispyngntkypqkfqgrvtmttdtstntaymelrslrsddtav | | |
| | yycardlelgggfdywgqgtlvtvss | | |
| 44 | qsaltqpasvsgspgqsitisctgtssdvgsynlvswyqqhpdkapk | #8 VL | |
| | fmiyegtkrpsgvsnrfsgsksgntasltisglqaedeadyyccsyag | | |
| | nstwvfgggtkltvl | | |
| 45 | EIVLTQSPGTLSLSPGERATLSC | #3 FR1 VL | Chothia, AbM, |
| | | | Kabat |
| 46 | WYQQKPGQAPRLLIY | #3 FR2 VL | Chothia, AbM, |
| | | | Kabat |
| 47 | GIPDRFSGSGSGTDFTLTISRLEPEDSAVYYC | #3 FR3 VL | Chothia, AbM, |
| | | | Kabat |
| 48 | FGQGTKLEIK | #3 FR4 VL | Chothia, AbM, |
| | | | Kabat, IMGT |
| 49 | EVQLVESGGGLVQPGGSLRLSCAAS | #3 FR1 VH | Chothia, AbM, |
| | | | IMGT |
| 50 | YMTWVRQAPGKGLEWVSVI | #3 FR2 VH | Chothia |
| 51 | TFYADSVRGRFTISRDNSKNTLYLQMNSLRAEDT | #3 FR3 VH | Chothia |
| | AVYYCAR | | |
| 52 | WGQGTMVTVSS | #3 FR4 VH | Chothia, AbM, |
| | | | Kabat, IMGT |
| 53 | QSALTQPASVSGSPGQSITISC | #8 FR1 VL | Chothia, AbM, |
| | | | Kabat |
| 54 | WYQQHPDKAPKFMIY | #8 FR2 VL | Chothia, AbM, |
| | | | Kabat |
| 55 | GVSNRFSGSKSGNTASLTISGLQAEDEADYYC | #8 FR3 VL | Chothia, AbM, |
| | | | Kabat |
| 56 | FGGGTKLTVL | #8 FR4 VL | Chothia, AbM, |
| | | | Kabat, IMGT |
| 57 | QVQLVQSGAEVKKPGASVKVSCKAS | #8 FR1 VH | Chothia, AbM, |
| | | | IMGT |
| 58 | GISWVRQAPGQGLEWMGWI | #8 FR2 VH | Chothia |
| 59 | TKYPQKFQGRVTMTTDTSTNTAYMELRSLRSDD | #8 FR3 VH | Chothia |
| | TAVYYCAR | | |
| 60 | WGQGTLVTVSS | #8 FR4 VH | Chothia, AbM, |
| | | | Kabat, IMGT |
| 61 | EIVLTQSPGTLSLSPGERATLSCRASQSV | #3 FR1 VL | Contact |
| 62 | EIVLTQSPGTLSLSPGERATLSCRAS | #3 FR1 VL | IMGT |
| 63 | QQKPGQAPR | #3 FR2 VL | Contact |
| 64 | LAWYQQKPGQAPRLLIY | #3 FR2 VL | IMGT |
| 65 | TGIPDRFSGSGSGTDFTLTISRLEPEDSAVYYC | #3 FR3 VL | Contact |
| 66 | SSRATGIPDRFSGSGSGTDFTLTISRLEPEDSAVY | #3 FR3 VL | IMGT |
| | YC | | |
| 67 | TFGQGTKLEIK | #3 FR4 VL | Contact |
| 68 | EVQLVESGGGLVQPGGSLRLSCAASGITVS | #3 FR1 VH | Kabat |
| 69 | EVQLVESGGGLVQPGGSLRLSCAASGITV | #3 FR1 VH | Contact |
| 70 | WVRQAPGKGLEWVS | #3 FR2 VH | AbM, Kabat |
| 71 | WVRQAPGKGLE | #3 FR2 VH | Contact |
| 72 | MTWVRQAPGKGLEWVSV | #3 FR2 VH | IMGT |
| 73 | YADSVRGRFTISRDNSKNTLYLQMNSLRAEDTAV | #3 FR3 VH | AbM |
| | YYCAR | | |
| 74 | RFTISRDNSKNTLYLQMNSLRAEDTAVYYCAR | #3 FR3 VH | Kabat |
| 75 | YADSVRGRFTISRDNSKNTLYLQMNSLRAEDTAV | #3 FR3 VH | Contact |
| | YYC | | |
| 76 | FYADSVRGRFTISRDNSKNTLYLQMNSLRAEDTA | #3 FR3 VH | IMGT |
| | VYYC | | |
| 77 | IWGQGTMVTVSS | #3 FR4 VH | Contact |
| 78 | QSALTQPASVSGSPGQSITISCTGTSSD | #8 FR1 VL | Contact |
| 79 | QSALTQPASVSGSPGQSITISCTGT | #8 FR1 VL | IMGT |
| 80 | QQHPDKAPK | #8 FR2 VL | Contact |
| 81 | VSWYQQHPDKAPKFMIY | #8 FR2 VL | IMGT |
| 82 | SGVSNRFSGSKSGNTASLTISGLQAEDEADYYC | #8 FR3 VL | Contact |
| 83 | TKRPSGVSNRFSGSKSGNTASLTISGLQAEDEAD | #8 FR3 VL | IMGT |
| | YYC | | |
| 84 | VFGGGTKLTVL | #8 FR4 VL | Contact |
| 85 | QVQLVQSGAEVKKPGASVKVSCKASGYTFS | #8 FR1 VH | Kabat |
| 86 | QVQLVQSGAEVKKPGASVKVSCKASGYTF | #8 FR1 VH | Contact |
| 87 | WVRQAPGQGLEWMG | #8 FR2 VH | AbM, Kabat |
| 88 | WVRQAPGQGLE | #8 FR2 VH | Contact |
| 89 | ISWVRQAPGQGLEWMGW | #8 FR2 VH | IMGT |
| 90 | YPQKFQGRVTMTTDTSTNTAYMELRSLRSDDTA | #8 FR3 VH | AbM |
| | VYYCAR | | |
| 91 | RVTMTTDTSTNTAYMELRSLRSDDTAVYYCAR | #8 FR3 VH | Kabat |
| 92 | YPQKFQGRVTMTTDTSTNTAYMELRSLRSDDTA | #8 FR3 VH | Contact |
| | VYYC | | |
| 93 | KYPQKFQGRVTMTTDTSTNTAYMELRSLRSDDT | #8 FR3 VH | IMGT |
| | AVYYC | | |
| 94 | YWGQGTLVTVSS | #8 FR4 VH | Contact |
| 95 | CTA CAC GAC GCT CTT CCG ATC TAG CAGT | primer | |
| | GGT ATC AAC GCA | | |
| 96 | CTA CAC GAC GCT CTT CCG ATC T AGC | primer | |

### REFERENCES

1. Prevost J, Finzi A. 2021. The great escape? SARS-COV-2 variants evading neutralizing responses. Cell Host Microbe 29:322-324.
2. Ullah I, Prevost J, Ladinsky M S, Stone H, Lu M, Anand S P, Beaudoin-Bussieres G, Benlarbi M, Ding S, Gasser R, Fink C, Chen Y, Tauzin A, Goyette G, Bourassa C, Medjahed H, Mack M, Chung K, Wilen C B, Dekaban G A, Dikeakos J D, Bruce E A, Kaufmann D E, Stamatatos L, McGuire A, Richard J, Pazgier M, Bjorkman P, Mothes W, Finzi A, Kumar P, Uchil P D. 2021. Live imaging of SARS-COV-2 infection in mice reveals neutralizing antibodies require Fc function for optimal efficacy. doi: 10.1101/2021.03.22.436337% J bioRxiv: 2021.03.22.436337.
3. Stamatatos L, Czartoski J, Wan Y H, Homad L J, Rubin V, Glantz H, Neradilek M, Seydoux E, Jennewein M F, MacCamy A J, Feng J, Mize G, De Rosa S C, Finzi A, Lemos M P, Cohen K W, Moodie Z, McElrath M J, McGuire A T. 2021. mRNA vaccination boosts cross-variant neutralizing antibodies elicited by SARS-COV-2 infection. Science doi: 10.1126/science.abg9175.
4. Graham C, Seow J, Huettner I, Khan H, Kouphou N, Acors S, Winstone H, Pickering S, Galao R P, Dupont L, Lista M J, Jimenez-Guardeno J M, Laing A G, Wu Y, Joseph M, Muir L, van Gils M J, Ng W M, Duyvesteyn H M E, Zhao Y, Bowden T A, Shankar-Hari M, Rosa A, Cherepanov P, McCoy L E, Hayday A C, Neil S J D, Malim M H, Doores K J. 2021. Neutralization potency of monoclonal antibodies recognizing dominant and subdominant epitopes on SARS-CoV-2 Spike is impacted by the B.1.1.7 variant. Immunity doi: 10.1016/j.immuni.2021.03.023.
5. Jennewein M F, MacCamy A J, Akins N R, Feng J, Homad L J, Hurlburt N K, Seydoux E, Wan Y H, Stuart A B, Edara W, Floyd K, Vanderheiden A, Mascola J R, Doria-Rose N, Wang L, Yang E S, Chu H Y, Torres J L, Ozorowski G, Ward A B, Whaley R E, Cohen K W, Pancera M, McElrath M J, Englund J A, Finzi A, Suthar M S, McGuire A T, Stamatatos L. 2021. Isolation and Characterization of Cross-Neutralizing Coronavirus Antibodies from COVID-19+Subjects. bioRxiv doi: 10.1101/2021.03.23.436684.
6. Zhou P, Yuan M, Song G, Beutler N, Shaabani N, Huang D, He W-t, Zhu X, Callaghan S, Yong P, Anzanello F, Peng L, Ricketts J, Parren M, Garcia E, Rawlings SA, Smith D M, Nemazee D, Teijaro J R, Rogers T F, Wilson I A, Burton D R, Andrabi R. 2021. A protective broadly cross-reactive human antibody defines a conserved site of vulnerability on beta-coronavirus spikes. doi: 10.1101/2021.03.30.437769% J bioRxiv: 2021.03.30.437769.
7. Ullah I, Prevost J, Ladinsky M S, Stone H, Lu M, Anand SP, Beaudoin-Bussieres G, Benlarbi M, Ding S, Gasser R, Fink C, Chen Y, Tauzin A, Goyette G, Bourassa C, Medjahed H, Mack M, Chung K, Wilen C B, Dekaban G A, Dikeakos J D, Bruce E A, Kaufmann D E, Stamatatos L, McGuire A T, Richard J, Pazgier M, Bjorkman P J, Mothes W, Finzi A, Kumar P, Uchil P D. 2021. Live imaging of SARS-COV-2 infection in mice reveals neutralizing antibodies require Fc function for optimal efficacy. bioRxiv doi: 10.1101/2021.03.22.436337.
8. Hoffmann M, Kleine-Weber H, Schroeder S, Kruger N, Herrler T, Erichsen S, Schiergens T S, Herrler G, Wu N H, Nitsche A, Muller M A, Drosten C, Pohlmann S. 2020. SARS-CoV-2 Cell Entry Depends on ACE2 and TMPRSS2 and Is Blocked by a Clinically Proven Protease Inhibitor. Cell 181:271-280 e8.
9. Prevost J, Gasser R, Beaudoin-Bussieres G, Richard J, Duerr R, Laumaea A, Anand S P, Goyette G, Benlarbi M, Ding S, Medjahed H, Lewin A, Perreault J, Tremblay T, Gendron-Lepage G, Gauthier N, Carrier M, Marcoux D, Piche A, Lavoie M, Benoit A, Loungnarath V, Brochu G, Haddad E, Stacey H D, Miller M S, Desforges M, Talbot P J, Maule G T G, Cote M, Therrien C, Serhir B, Bazin R, Roger M, Finzi A. 2020. Cross-Sectional Evaluation of Humoral Responses against SARS-COV-2 Spike. Cell Rep Med 1:100126.
10. Wrapp D, Wang N, Corbett K S, Goldsmith J A, Hsieh C L, Abiona O, Graham B S, Mclellan J S. 2020. Cryo-EM structure of the 2019-nCOV spike in the prefusion conformation. Science 367:1260-1263.
11. Beaudoin-Bussieres G, Laumaea A, Anand S P, et al. Decline of Humoral Responses against SARS-COV-2 Spike in Convalescent Individuals. mBio 2020;11.
12. Lu M, Uchil PD, Li W, et al. Real-Time Conformational Dynamics of SARS-COV-2 Spikes on Virus Particles. Cell Host Microbe 2020;28:880-891 e8.
13. Ding S, Laumaea A, Benlarbi M, et al. Antibody Binding to SARS-COV-2 S Glycoprotein Correlates with but Does Not Predict Neutralization. Viruses 2020; 12.
14. Anand S P, Chen Y, Prevost J, et al. Interaction of Human ACE2 to Membrane-Bound SARS-COV-1 and SARS-COV-2 S Glycoproteins. Viruses 2020;12.
15. Simone Picelli, Omid R Faridani, Asa K Björklund, Gosta Winberg, Sven Sagasser, Rickard Sandberg. Full-length RNA-seq from single cells using Smart-seq2. Nat Protoc. 2014 Jan;9(1): 171-81. doi: 10.1038/nprot.2014.006. Epub 2014 Jan. 2.
16. Wang E, Wang LC, Tsai CY, et al. Generation of Potent T-cell Immunotherapy for Cancer Using DAP12-Based, Multichain, Chimeric Immunoreceptors. Cancer Immunol Res 2015;3:815-26.
17. Colamartino A B L, Lemieux W, Bifsha P, et al. Efficient and Robust NK-Cell Transduction With Baboon Envelope Pseudotyped Lentivector. Frontiers in Immunology 2019;10.
18. Denman C J, Senyukov W, Somanchi S S, et al. Membrane-bound IL-21 promotes sustained ex vivo proliferation of human natural killer cells. PLOS One 2012;7: e30264.

1. An antibody or an antigen binding fragment thereof comprises one of the following combinations of complementarity determining regions (CDRs):
(a) a light chain CDR1 (CDR-L1) comprising an amino acid sequence having at least 70% identity with the sequence RASQSVSSSYLA (SEQ ID NO:14); a CDR-L2 comprising an amino acid sequence having at least 70% identity with the sequence GASSRAT (SEQ ID NO:17); a CDR-L3 comprising an amino acid sequence having at least 70% identity with the sequence QQYGSSYT (SEQ ID NO: 19); a heavy chain CDR1 (CDR-H1) comprising an amino acid sequence having at least 70% identity with the sequence GITVSSN (SEQ ID NO:1); a CDR-H2 comprising an amino acid sequence having at least 70% identity with the sequence YSGGS (SEQ ID NO:6); and a CDR-H3 comprising an amino acid sequence having at least 70% identity with the sequence DLEMAGAFDI (SEQ ID NO:11); or
(b) a CDR-L1 comprising an amino acid sequence having at least 70% identity with the sequence TGTSSDVGSYNLVS (SEQ ID NO:36); a CDR-L2 comprising an amino acid sequence having at least 70% identity with the sequence EGTKRPS (SEQ ID NO: 39); a CDR-L3 comprising an amino acid sequence having at least 70% identity with the sequence CSYAGNSTWV (SEQ ID NO:41); a CDR-H1 comprising an amino acid sequence having at least 70% identity with the sequence GYTFSSY (SEQ ID NO:23); a CDR-H2 comprising an amino acid sequence having at least 70% identity with the sequence SPYNGN (SEQ ID NO:28); and a CDR-H3 comprising an amino acid sequence having at least 70% identity with the sequence DLELGGGFDY (SEQ ID NO:33).

2. The antibody or antigen binding fragment thereof of claim 1, which comprises the following combinations of complementarity determining regions (CDRs):
a CDR-L1 comprising the sequence RASQSVSSSYLA (SEQ ID NO:14); a CDR-L2 comprising the sequence GASSRAT (SEQ ID NO:17); a CDR-L3 comprising the sequence QQYGSSYT (SEQ ID NO:19); a CDR-H1 comprising the sequence GITVSSN (SEQ ID NO:1); a CDR-H2 comprising the sequence YSGGS (SEQ ID NO:6); and a CDR-H3 comprising the sequence DLEMAGAFDI (SEQ ID NO:11).

3. The antibody or antigen binding fragment thereof of claim 1, which comprises the following combinations of complementarity determining regions (CDRs):
a CDR-L1 comprising the sequence TGTSSDVGSYNLVS (SEQ ID NO:36); a CDR-L2 comprising the sequence EGTKRPS (SEQ ID NO:39); a CDR-L3 comprising the sequence CSYAGNSTWV (SEQ ID NO:41); a CDR-H1 comprising the sequence GYTFSSY (SEQ ID NO:23); a CDR-H2 comprising the sequence SPYNGN (SEQ ID NO: 28); and a CDR-H3 comprising the sequence DLELGGGFDY (SEQ ID NO:33).

4. The antibody or antigen binding fragment thereof of claim 1, which further comprises the following light chain framework regions (FRs):
(i) a light chain FR1 comprising an amino acid sequence having at least 50% identity with the sequence EIVLTQSPGTLSLSPGERATLSC (SEQ ID NO:45) or QSALTQPASVSGSPGQSITISC (SEQ ID NO:53); (ii) a light chain FR2 comprising an amino acid sequence having at least 50% identity with the sequence WYQQKPGQAPRLLIY (SEQ ID NO:46) or WYQQHPDKAPKFMIY (SEQ ID NO:54); (iii) a light chain FR3 comprising an amino acid sequence having at least 50% identity with the sequence GIPDRFSGSGSGTDFTLTISRLEPEDSAVYYC (SEQ ID NO:47) or GVSNRFSGSKSGNTASLTISGLQAEDEADYYC (SEQ ID NO:55); (iv) a light chain FR4 comprising an amino acid sequence having at least 50% identity with the sequence FGQGTKLEIK (SEQ ID NO:48) or FGGGTKLTVL (SEQ ID NO:56); or (v) any combination of (i) to (iv).

5. The antibody or antigen binding fragment thereof of claim 4, which comprises the following FRs:
(i) a light chain FR1 comprising the sequence EIVLTQSPGTLSLSPGERATLSC (SEQ ID NO:45); (ii) a light chain FR2 comprising the sequence WYQQKPGQAPRLLIY (SEQ ID NO:46); (iii) a light chain FR3 comprising the sequence GIPDRFSGSGSGTDFTLTISRLEPEDSAVYYC (SEQ ID NO:47); and (iv) a light chain FR4 comprising the sequence FGQGTKLEIK (SEQ ID NO:48).

6. The antibody or antigen binding fragment thereof of claim 4, which comprises the following light chain FRs:
(i) a light chain FR1 comprising the sequence QSALTQPASVSGSPGQSITISC (SEQ ID NO:53); (ii) a light chain FR2 comprising the sequence WYQQHPDKAPKFMIY (SEQ ID NO:54); (iii) a light chain FR3 comprising the sequence GVSNRFSGSKSGNTASLTISGLQAEDEADYYC (SEQ ID NO:55); and (iv) a light chain FR4 comprising the sequence FGGGTKLTVL (SEQ ID NO:56).

7. The antibody or antigen binding fragment thereof of claim 1, which further comprises the following heavy chain FRs:
(i) a heavy chain FR1 comprising an amino acid sequence having at least 50% identity with the sequence EVOLVESGGGLVQPGGSLRLSCAAS (SEQ ID NO:49) or QVQLVQSGAEVKKPGASVKVSCKAS (SEQ ID NO:57); (ii) a heavy chain FR2 comprising an amino acid sequence having at least 50% identity with the sequence YMTWVRQAPGKGLEWVSVI (SEQ ID NO:50) or GISWVRQAPGQGLEWMGWI (SEQ ID NO: 58); (iii) a heavy chain FR3 comprising an amino acid sequence having at least 50% identity with the sequence TFYADSVRGRFTISRDNSKNTLYLQMNSLRAEDTAVYYCAR (SEQ ID NO:51) or TKYPQKFQGRVTMTTDTSTNTAYMELRSLRSDDTAVYYCAR (SEQ ID NO:59); (iv) a heavy chain FR4 comprising an amino acid sequence having at least 50%, 60%, 70%, 75%, 80%, 85%, 90% or 95% identity with the sequence WGQGTMVTVSS (SEQ ID NO: 52) or WGQGTLVTVSS (SEQ ID NO:60); or (v) any combination of (i) to (iv).

8. The antibody or antigen binding fragment thereof of claim 7, which further comprises the following heavy chain FRs:
(i) a heavy chain FR1 comprising the sequence EVOLVESGGGLVQPGGSLRLSCAAS (SEQ ID NO:49); (ii) a heavy chain FR2 comprising the sequence GISWVRQAPGQGLEWMGWI (SEQ ID NO:50); (iii) a heavy chain FR3 comprising the sequence TKYPQKFQGRVTMTTDTSTNTAYMELRSLRSDDTAVYYCAR (SEQ ID NO:51); and (iv) a heavy chain FR4 comprising the sequence WGQGTMVTVSS (SEQ ID NO:52).

9. The antibody or antigen binding fragment thereof of claim 7, which further comprises the following heavy chain FRs:
(i) a heavy chain FR1 comprising the sequence QVQLVQSGAEVKKPGASVKVSCKAS (SEQ ID NO:57); (ii) a heavy chain FR2 comprising the sequence YMTWVRQAPGKGLEWVSVI (SEQ ID NO:58); (iii) a heavy chain FR3 comprising the sequence TFYADSVRGRFTISRDNSKNTLYLQMNSLRAEDTAVYYCAR (SEQ ID NO:59); and (iv) a heavy chain FR4 comprising the sequence WGQGTLVTVSS (SEQ ID NO:60).

10. (canceled)

11. The antibody or antigen binding fragment thereof of claim 1, which comprises a variable light chain comprising the following sequence:
EIVLTQSPGTLSLSPGERATLSCRASQSVSSSYLAWYQQKPGQAPRLLIYGASSRATGI PDRFSGSGSGTDFTLTISRLEPEDSAVYYCQQYGSSYTFGQQTKLEIK (SEQ ID NO: 22); and
a variable heavy chain comprising the following sequence:
(SEQ ID NO: 21)
EVQLVESGGGLVQPGGSLRLSCAASGITVSSNYMTWVRQAPGKGLEWVS
VIYSGGSTFYADSVRGRFTISRDNSKNTLYLQMNSLRAEDTAVYYCARD
LEMAGAFDIWGQGTMVTVSS.

12. The antibody or antigen binding fragment thereof of claim 1, which comprises a variable light chain comprising the following sequence:
QSALTQPASVSGSPGQSITISCTGTSSDVGSYNLVSWYQQHPDKAPKFMIYEGTKRPS GVSNRFSGSKSGNTASLTISGLQAEDEADYYCCSYAGNSTWVFGGGTKLTVL (SEQ ID NO: 44); and
a variable heavy chain comprising the following sequence:
(SEQ ID NO: 43)
QVQLVQSGAEVKKPGASVKVSCKASGYTFSSYGISWVRQAPGQGLEWMG
WISPYNGNTKYPQKFQGRVTMTTDTSTNTAYMELRSLRSDDTAVYYCAR
DLELGGGFDYWGQGTLVTVSS.

13-15. (canceled)

16. A conjugate or a chimeric antigen receptor (CAR) comprising the antibody or antigen binding fragment thereof of claim 1.

17. A nucleic acid comprising a sequence encoding the light and/or heavy chain of the antibody or antigen binding fragment thereof of claim 1.

18. A host cell comprising the nucleic acid of claim 17.

19. A pharmaceutical composition comprising the antibody or antigen binding fragment thereof of claim 1, or one or more nucleic acids comprising a sequence encoding said antibody or antigen binding fragment thereof, and a pharmaceutically acceptable excipient.

20. The pharmaceutical composition of claim 19, wherein the pharmaceutical composition is in the form of an aerosol or an injectable solution.

21. A method for preventing or treating a SARS-COV-2 infection or a related disease (COVID-19) in a subject in need thereof, the method comprising administering to the subject an effective amount of (i) the antibody or antigen-binding fragment thereof of claim 1, (ii) a conjugate or a chimeric antigen receptor (CAR) comprising said antibody or antigen binding fragment thereof, (iii) one or more nucleic acids comprising a sequence encoding said antibody or antigen binding fragment thereof; or (iv) a pharmaceutical composition comprising said antibody or antigen binding fragment thereof or said one or more nucleic acids.

22. A method for reducing the risk of developing Coronavirus disease 2019 (COVID-19) or the severity of COVID-19 in a subject infected by SARS-CoV-2, the method comprising administering to the subject an effective amount of (i) the antibody or antigen-binding fragment thereof of claim 1, (ii) a conjugate or a chimeric antigen receptor (CAR) comprising said antibody or antigen binding fragment thereof, (iii) one or more nucleic acids comprising a sequence encoding said antibody or antigen binding fragment thereof; or (iv) a pharmaceutical composition comprising said antibody or antigen binding fragment thereof or said one or more nucleic acids.

23. A method for blocking the entry of SARS-COV-2 in an ACE2-expressing cell, the method comprising contacting the cell and/or the virus with an effective amount of the antibody or antigen-binding fragment thereof of (i) the antibody or antigen-binding fragment thereof of claim 1, (ii) a conjugate or a chimeric antigen receptor (CAR) comprising said antibody or antigen binding fragment thereof, (iii) one or more nucleic acids comprising a sequence encoding said antibody or antigen binding fragment thereof; or (iv) a pharmaceutical composition comprising said antibody or antigen binding fragment thereof or said one or more nucleic acids.

24. (canceled)

25. The method of claim 21, wherein the antibody, antigen-binding fragment thereof, conjugate, CAR, one or more nucleic acids or pharmaceutical composition is administered with at least one additional anti-SARS-COV-2 antibody or antigen-binding fragment thereof.

26-61. (canceled)
